(19) United States
(12) Patent Application Publication
(10) Pub. No.: US 2016/0130259 A1
(43) Pub. Date: May 12, 2016

(54) SUBSTITUTED BENZOFURAN COMPOUNDS AND METHODS OF USE THEREOF FOR THE TREATMENT OF VIRAL DISEASES

(71) Applicant: Merck Sharp & Dohme Corp., Rahway, NJ (US)

(72) Inventors: Hong Liu, Hillsborough, NJ (US); Xing Dai, Cranford, NJ (US); Anandan Palani, Bridgewater, NJ (US); Shuwen He, Fanwood, NJ (US); Ravi Nargund, East Brunswick, NJ (US); Dong Xiao, Warren, NJ (US); Qun Dang, Westfield, NJ (US); Xuanjia Peng, Shanghai (CN); Peng Li, Shanghai (CN)

(73) Assignee: Merck Sharp & Dohme Corp., Rahway, NJ (US)

(21) Appl. No.: 14/898,250

(22) PCT Filed: Jun. 19, 2014
(86) PCT No.: PCT/US2014/043069

§ 371 Date: Dec. 14, 2015

(30) Foreign Application Priority Data
Jun. 24, 2013 (CN) PCT/CN2013/000747

(51) Int. Cl. C07D 405/14; C07D 491/048; C07D 498/04; C07D 471/04; C07D 471/14; A61K 31/4985; A61K 31/4439; A61K 31/444; A61K 31/501; A61K 31/497; A61K 31/506; C07D 413/14; A61K 45/06

(52) U.S. Cl. CPC C07D 405/14; C07D 413/14; C07D 491/048; C07D 498/04; C07D 471/04; C07D 471/14; A61K 45/06; A61K 31/4439; A61K 31/444; A61K 31/501; A61K 31/497; A61K 31/506; A61K 31/4985

(57) ABSTRACT

The present invention relates to compounds of formula I that are useful as hepatitis C virus (HCV) NS5B polymerase inhibitors, the synthesis of such compounds, and the use of such compounds for inhibiting HCV NS5B polymerase activity, for treating or preventing HCV infections and for inhibiting HCV viral replication and/or viral production in a cell-based system.

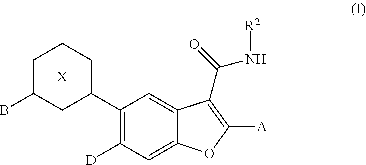

### FIELD OF THE INVENTION

The present disclosure relates to compounds that are useful as inhibitors of the hepatitis C virus (HCV) NS5B (non-structural protein 5B) polymerase, compositions comprising such compounds, the use of such compounds for treating HCV infection and/or reducing the likelihood or severity of symptoms of HCV infection, methods for inhibiting the function of the NS5B polymerase, and methods for inhibiting HCV viral replication and/or viral production.

### BACKGROUND OF THE INVENTION

Hepatitis C virus (HCV) infection is a major health problem that leads to chronic liver disease, such as cirrhosis and hepatocellular carcinoma, in a substantial number of infected individuals. Current treatments for HCV infection include immunotherapy with recombinant interferon-α alone or in combination with the nucleoside analog ribavirin.

Several virally-encoded enzymes are putative targets for therapeutic intervention, including a metalloprotease (NS2-3), a serine protease (NS3, amino acid residues 1-180), a helicase (NS3, full length), an NS3 protease cofactor (NS4A), a membrane protein (NS4B), a zinc metalloprotein (NS5A) and an RNA-dependent RNA polymerase (NS5B).

One identified target for therapeutic intervention is HCV NS5B polymerase. Sven-Erik Behrens et al., Identification and properties of the RNA-dependent RNA polymerase of hepatitis C virus, 15(1) EMBO J. 12-22 (1996). Antagonists of NS5B activity are inhibitors of HCV replication. Steven S. Carroll et al., Inhibition of Hepatitis C Virus RNA Replication by 2′-Modified Nucleoside Analogs, 278(14) J. BIOL. CHEM. 11979-84 (2003).

There is a clear and long-felt need to develop effective therapeutics for treatment of HCV infection. Specifically, there is a need to develop compounds that inhibit HCV viral replication and that would be useful for treating HCV-infected patients.

### SUMMARY OF THE INVENTION

The present disclosure relates to novel compounds of formula I and pharmaceutically acceptable salts thereof. These compounds are useful, either as compounds or their pharmaceutically acceptable salts (when appropriate), in the inhibition of HCV (hepatitis C virus) NS5B (non-structural 5B) polymerase, the prevention or treatment of one or more of the symptoms of HCV infection, the inhibition of HCV viral replication and/or HCV viral production, and/or as pharmaceutical composition ingredients. As pharmaceutical composition ingredients, these compounds and their salts may be the primary active therapeutic agent, and, when appropriate, may be combined with other therapeutic agents including but not limited to other HCV antivirals, anti-infectives, immunomodulators, antibiotics or vaccines, as well as the present Standard of Care treatment options for HCV.

In one aspect, the present invention relates to a compound of formula I:

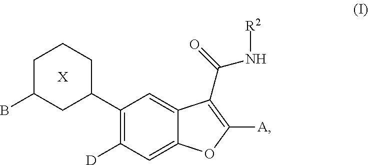

or a pharmaceutically acceptable salt thereof,
wherein:

X is

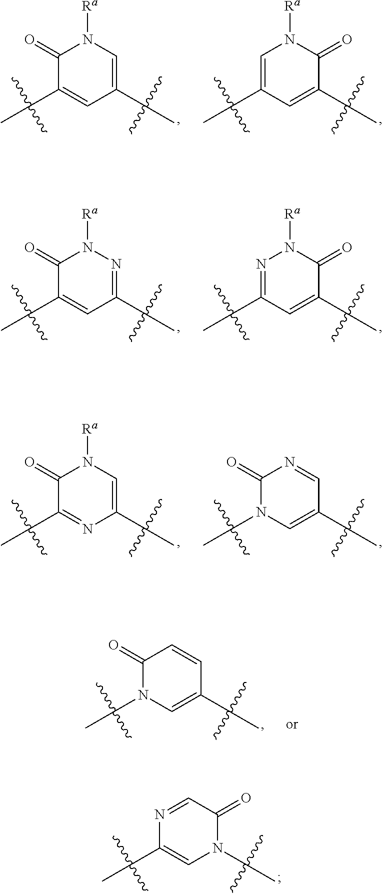

B is a) Ar; or
b) —C(═O)NHCR⁵R⁶Ar; or

X together with B is

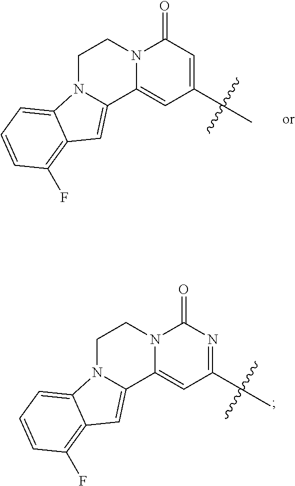 [SMILES: *c1cc2n(c(=O)c1)CCn1c-2cc2c(F)cccc21 ; *c1cc2n(c(=O)n1)CCn1c-2cc2c(F)cccc21]

Ar is an aromatic ring system selected from:
(i) 5-6 membered monocyclic ring with 0, 1, or 2 N ring atoms, optionally substituted with halo or fluorophenyl; and
(ii) 9-10 membered bicyclic rings with 0, 1, 2 or 3 heteroatom ring atoms selected from N and O, which is optionally substituted with 1 or 2 substituents independently selected from C₁-C₆ alkyl, F, cyano, oxo, and alkylalkoxy;

A is fluorophenyl;

D is H or NR³SO₂R⁴;

R^{a} is C₁-C₆ alkyl or C₁-C₆ haloalkyl;

R², R³, and R⁴ are independently C₁-C₆ alkyl;

R⁵ is hydrogen, C₁-C₆ alkyl, or C₁-C₆ hydroxyalkyl;

R⁶ is hydrogen; or

R⁵ and R⁶ together with the carbon to which they are attached form cyclopropyl.

The present invention also includes pharmaceutical compositions containing a compound of the present invention and methods of preparing such pharmaceutical compositions. The present invention further includes methods of treating or reducing the likelihood or severity of HCV infection, methods for inhibiting the activity of the NS5B polymerase, and methods for inhibiting HCV viral replication and/or viral production.

Other embodiments, aspects and features of the present invention are either further described in or will be apparent from the ensuing description, examples and appended claims.

### DETAILED DESCRIPTION OF THE INVENTION

The present invention includes compounds of formula I above, and pharmaceutically acceptable salts thereof. The compounds of formula I are HCV NS5B polymerase inhibitors.

In a first embodiment of the invention, R², R³ and R⁴ are methyl, and the other groups are as provided in the general formula above.

In a second embodiment of the invention, D is N(CH₃)SO₂CH₃ and the other groups are as provided in the general formula above, or as in the first embodiment.

In a third embodiment of the invention, each halo is F, and the other groups are as provided in the general formula above, or as in the first or second embodiments.

In a fourth embodiment of the invention, R^{a} is methyl or

—CHF₂ and the other groups are as provided in the general formula above, or as in the first through third embodiments.

In a fifth embodiment of the invention, R⁵ is hydrogen, methyl or —CH₂OH, or R⁵ and R⁶ together with the carbon to which they are attached form cyclopropyl, and the other groups are as provided in the general formula above, or as in the first through fourth embodiments.

In a sixth embodiment of the invention, the compound of the invention has the formula:

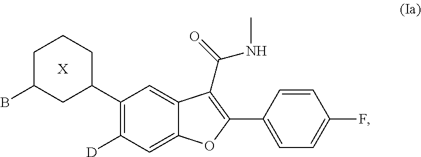

or a pharmaceutically acceptable salt thereof, and the other groups are as provided in the general formula above, or as in the first through fifth embodiments.

In a seventh embodiment of the invention, B is fluorophenyl; pyrazole substituted with fluorophenyl; —C(═O)NHCH(CH₃)-fluorophenyl; —C(═O)NHCH(CH₂OH)-fluorophenyl; C(═O)NHCH₂-fluoropyridine; —C(═O)NH-cyclopropyl-phenyl; or —C(═O)NH-cyclopropyl-fluorophenyl, and the other groups are as provided in the general formula above, or as in the first through sixth embodiments.

In an eighth embodiment of the invention, B is indole substituted 1 or 2 substituents selected from H, F, cyano, and —CH₂CH₂OCH₃; benzooxazole; isoindolinone substituted with F; furopyridine; oxaxolopyridine; pyrrolopryidine; naphthalene; or —C(═O)NH-cyclopropyl-naphthyridine, and the other groups are as provided in the general formula above, or as in the first through sixth embodiments.

In certain aspects of the invention, a fluorophenyl is para-fluorophenyl.

In another embodiment of the invention, the compound of the invention is selected from the exemplary species depicted in Examples 1-29 shown below, and pharmaceutically acceptable salts thereof.

Other embodiments of the present invention include the following:

(a) A pharmaceutical composition comprising an effective amount of a compound of formula I and a pharmaceutically acceptable carrier.

(b) The pharmaceutical composition of (a), further comprising a second therapeutic agent selected from the group consisting of HCV antiviral agents, immunomodulators, and anti-infective agents.

(c) The pharmaceutical composition of (b), wherein the HCV antiviral agent is an antiviral selected from the group consisting of direct inhibitors of HCV, including but not limited to NS3 and NS3/4A protease inhibitors, NS5A inhibitors and HCV NS5B polymerase inhibitors.

(d) A pharmaceutical combination that is (i) a compound of formula I and (ii) a second therapeutic agent selected from the group consisting of HCV antiviral agents, immunomodulators, and anti-infective agents; wherein the compound of formula I and the second therapeutic agent are each employed in an amount that renders the combination effective for inhibiting HCV NS5B activity, or for inhibiting HCV viral replication, or for treating HCV infection and/or reducing the likelihood or severity of symptoms of HCV infection.

(e) The combination of (d), wherein the HCV antiviral agents are one or more antiviral agents selected from the group consisting of direct inhibitors of HCV, including but not limited to NS3 and NS3/4A protease inhibitors, NS5A inhibitors and HCV NS5B polymerase inhibitors.

(f) A use of a compound of formula I in the preparation of a medicament for inhibiting HCV NS5B activity in a subject in need thereof.

(g) A use of a compound of formula I in the preparation of a medicament for preventing and/or treating infection by HCV in a subject in need thereof.

(h) A method of treating HCV infection and/or reducing the likelihood or severity of symptoms of HCV infection in a subject in need thereof, which comprises administering to the subject an effective amount of a compound of formula I.

(i) The method of (h), wherein the compound of formula I is administered in combination with an effective amount of at least one second therapeutic agent selected from the group consisting of HCV antiviral agents, immunomodulators, and anti-infective agents.

(j) The method of (i), wherein the HCV antiviral agent is an antiviral selected from the group consisting of direct inhibitors of HCV, including but not limited to NS3 and NS3/4A protease inhibitors, NS5A inhibitors and HCV NS5B polymerase inhibitors.

(k) A method of inhibiting HCV viral replication and/or HCV viral production in a cell-based system, which comprises administering to the subject an effective amount of a compound of formula I in combination with an effective amount of at least one second therapeutic agent selected from the group consisting of HCV antiviral agents, immunomodulators, and anti-infective agents.

(l) The method of (k), wherein the HCV antiviral agent is an antiviral selected from the group consisting of direct inhibitors of HCV, including but not limited to NS3 and NS3/4A protease inhibitors, NS5A inhibitors and HCV NS5B polymerase inhibitors.

(m) A method of inhibiting HCV NS5B activity in a subject in need thereof, which comprises administering to the subject the pharmaceutical composition of (a), (b), or (c) or the combination of (d) or (e).

(n) A method of treating HCV infection and/or reducing the likelihood or severity of symptoms of HCV infection in a subject in need thereof, which comprises administering to the subject the pharmaceutical composition of (a), (b), or (c) or the combination of (d) or (e).

In the embodiments of the compounds and salts provided above, it is to be understood that each embodiment may be combined with one or more other embodiments, to the extent that such a combination provides a stable compound or salt and is consistent with the description of the embodiments. It is further to be understood that the embodiments of compositions and methods provided as (a) through (n) above are understood to include all embodiments of the compounds and/or salts, including such embodiments as result from combinations of embodiments.

Additional embodiments of the invention include the pharmaceutical compositions, combinations, uses and methods set forth in (a) through (n) above, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

The present invention also includes a compound of the present invention for use (i) in, (ii) as a medicament for, or (iii) in the preparation of a medicament for: (a) inhibiting HCV NS5B activity, or (b) inhibiting HCV viral replication, or (c) treating HCV infection and/or reducing the likelihood or severity of symptoms of HCV infection, or (d) use in medicine. In these uses, the compounds of the present invention can optionally be employed in combination with one or more second therapeutic agents selected from HCV antiviral agents, anti-infective agents, and immunomodulators.

Chemical names, common names, and chemical structures may be used interchangeably to describe the same structure. If a chemical compound is referred to using both a chemical structure and a chemical name and an ambiguity exists between the structure and the name, the structure is understood to predominate.

As used herein, the term “administration” and variants thereof (e.g., “administering” a compound) in reference to a compound of the invention mean providing the compound to the individual in need of treatment. When a compound of the invention is provided in combination with one or more other active agents (e.g., antiviral agents useful for treating HCV infection), “administration” and its variants are each understood to include concurrent and sequential provision of the compound or salt and other agents.

As used herein, the term “alkoxy” refers to an “alkyl-O—” group. Alkoxy groups may be substituted as indicated.

The term “alkyl” refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 3 carbon atoms (C₁-C₃ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched.

The term “aryl” (or “aryl ring system”) refers to aromatic mono- and poly-carbocyclic ring systems wherein the individual carbocyclic rings in the polyring systems are fused or attached to each other via a single bond. As used herein, the term aryl includes aromatic mono- and poly-carbocyclic ring systems that include from 0 to 4 heteroatoms (non-carbon atoms) that are independently chosen from N, O and S. Suitable aryl groups include phenyl, naphthyl, biphenylenyl, pyridinyl, pyrimidinyl and pyrrolyl, as well as those discussed below. Aryl ring systems may include, where appropriate, an indication of the variable to which a particular ring atom is attached. Unless otherwise indicated, substituents to the aryl ring systems can be attached to any ring atom, provided that such attachment results in formation of a stable ring system.

The term “composition” is intended to encompass a product comprising the specified ingredients, as well as any product which results from combining the specified ingredients.

The term “compound” is intended to encompass chemical agents described by generic formula I in all forms. Such chemical agents can be present in different forms such as hydrates and solvates.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl, bicyclo[3.1.0]hexyl and adamantyl. The term “3 to 7-membered cycloalkyl” refers to a cycloalkyl group having from 3 to 7 ring carbon atoms. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

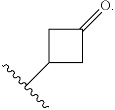 [SMILES: *C1CC(=O)C1]

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of one or more symptoms of the disease or condition being treated. In another embodiment, the effective amount is a “prophylactically effective amount” for reduction of the severity or likelihood of one or more symptoms of the disease or condition. In another embodiment, the effective amount is a “therapeutically effective amount” for inhibition of HCV viral replication and/or HCV viral production. The term also includes herein the amount of active compound sufficient to inhibit HCV NS5B activity and thereby elicit the response being sought (i.e., an “inhibition effective amount”). When the active compound (i.e., active ingredient) is administered as the salt, references to the amount of active ingredient are to the free acid or free base form of the compound.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “halogen” (or “halo”) refers to atoms of fluorine, chlorine, bromine and iodine (alternatively referred to as fluoro, chloro, bromo, and iodo).

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic and has 9 or 10 ring atoms. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. The term “heteroaryl” also encompasses any fused polycyclic ring system containing at least one ring heteroatom selected from N, O and S, wherein at least one ring of the fused polycyclic ring system is aromatic. For example, the term “9 to 10-membered bicyclic heteroaryl” encompasses a non-aromatic 5 membered heterocyclic ring that is fused to a benzene or pyridyl ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl. In another embodiment, a heteroaryl group comprises a 5- to 6-membered heteroaryl group fused to a benzene ring.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

As used herein, the term “oxo” or “═O” forms a carbonyl moiety with the carbon atom to which it is attached.

By “pharmaceutically acceptable” is meant that the ingredients of the pharmaceutical composition must be compatible with each other and not deleterious to the recipient thereof.

The term “preventing,” as used herein with respect to an HCV viral infection or HCV-virus related disorder, refers to reducing the likelihood of HCV infection.

The term “subject” (alternatively referred to herein as “patient”), as used herein, refers to an animal, preferably a mammal, most preferably a human.

The term “substituted” means that one or more hydrogens on the designated atom is replaced with a selection from the indicated group, provided that the designated atom's normal valency under the existing circumstances is not exceeded, and that the substitution results in a stable compound. Unless expressly stated to the contrary, substitution by a named substituent is permitted on any atom provided such substitution is chemically allowed and results in a stable compound. Combinations of substituents and/or variables are permissible only if such combinations result in stable compounds. A “stable” compound is a compound that can be prepared and isolated and whose structure and properties remain or can be caused to remain essentially unchanged for a period of time sufficient to allow use of the compound for the purposes described herein (e.g., therapeutic or prophylactic administration to a subject).

In the compounds of formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of formula I. For example, different isotopic forms of hydrogen (H) include protium (¹H) and deuterium (²H or D). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within formula I can be prepared without undue experimentation by conventional techniques well known to those skilled in the art or by processes analogous to those described in the Schemes and Examples herein using appropriate isotopically-enriched reagents and/or intermediates.

Unless expressly stated to the contrary, all ranges cited herein are inclusive. For example, a heteroaryl ring described as containing from “1 to 3 heteroatoms” means the ring can contain 1, 2, or 3 heteroatoms. It is also to be understood that any range cited herein includes within its scope all of the sub-ranges within that range. The oxidized forms of the heteroatoms N and S are also included within the scope of the present invention.

When any variable (for example, R¹ or R³) occurs more than one time in any constituent or in formula I or in any other formula depicting and describing compounds of the invention, its definition on each occurrence is independent of its definition at every other occurrence. Also, combinations of substituents and/or variables are permissible only if such combinations result in stable compounds.

Certain of the compounds of the present invention can have asymmetric centers and can occur as mixtures of stereoisomers, or as individual diastereomers, or enantiomers. All isomeric forms of these compounds, whether isolated or in mixtures, are within the scope of the present invention.

Certain of the compounds of the present invention can exist as tautomers. For the purposes of the present invention a reference to a compound of formula I is a reference to the compound per se, or to any one of its tautomers per se, or to mixtures of two or more tautomers.

The compounds of the present inventions are useful in the inhibition of HCV replication (e.g., HCV NS5B activity), the treatment of HCV infection and/or reduction of the likelihood or severity of symptoms of HCV infection. For example, the compounds of this invention are useful in treating infection by HCV after suspected past exposure to HCV by such means as blood transfusion, exchange of body fluids, bites, accidental needle stick, or exposure to patient blood during surgery.

The compounds of this invention are useful in the preparation and execution of screening assays for antiviral compounds. For example, the compounds of this invention are useful for identifying resistant HCV replicon cell lines harboring mutations within NS5B, which are excellent screening tools for more powerful antiviral compounds. Furthermore, the compounds of this invention are useful in establishing or determining the binding site of other antivirals to the HCV replicase.

The compounds of the present invention may be administered in the form of pharmaceutically acceptable salts. The term “pharmaceutically acceptable salt” refers to a salt that possesses the effectiveness of the parent compound and that is not biologically or otherwise undesirable (e.g., is neither toxic nor otherwise deleterious to the recipient thereof). Suitable salts include acid addition salts that may, for example, be formed by mixing a solution of the compound of the present invention with a solution of a pharmaceutically acceptable acid such as hydrochloric acid, sulfuric acid, acetic acid, trifluoroacetic acid, or benzoic acid. Many of the compounds of the invention carry an acidic moiety, in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

For the purposes of inhibiting HCV NS5B polymerase, treating HCV infection and/or reducing the likelihood or severity of symptoms of HCV infection and inhibiting HCV viral replication and/or HCV viral production, the compounds of the present invention, optionally in the form of a salt, can be administered by any means that produces contact of the active agent with the agent's site of action. They can be administered by one or more conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered by one or more of the following: orally, parenterally (including subcutaneous injections, intravenous, intramuscular, intrasternal injection or infusion techniques), by inhalation (such as in a spray form), or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as solubility aids. Injectable solutions can be prepared according to methods known in the art wherein the carrier comprises a saline solution, a glucose solution or a solution containing a mixture of saline and glucose. Further description of methods suitable for use in preparing pharmaceutical compositions of the present invention and of ingredients suitable for use in said compositions is provided in Remington's Pharmaceutical Sciences, 18^{th} edition (ed. A. R. Gennaro, Mack Publishing Co., 1990).

The compounds of this invention can be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, the compositions can be provided in the form of tablets or capsules containing 1.0 to 500 mg of the active ingredient, particularly 1, 5, 10, 15, 20, 25, 50, 75, 100, 150, 200, 250, 300, 400, and 500 mg of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, HCV viral genotype, viral resistance, and the host undergoing therapy.

As noted above, the present invention also relates to a method of inhibiting HCV NS5B activity, inhibiting HCV viral replication and/or HCV viral production, treating HCV infection and/or reducing the likelihood or severity of symptoms of HCV infection with a compound of the present invention in combination with one or more therapeutic agents and a pharmaceutical composition comprising a compound of the present invention and one or more therapeutic agents selected from the group consisting of a HCV antiviral agent, an immunomodulator, and an anti-infective agent. Such agents are described in detail below.

HCV polymerase inhibitors useful in the present compositions and methods include, but are not limited to, VP-19744 (Wyeth/ViroPharma), PSI-7851 (Pharmasset), RG7128 (Roche/Pharmasset), PSI-7977 (Pharmasset), PSI-938 (Pharmasset), PSI-879 (Pharmasset), PSI-661 (Pharmasset), PF-868554/filibuvir (Pfizer), VCH-759/VX-759 (ViroChem Pharma/Vertex), HCV-371 (Wyeth/VirroPharma), HCV-796 (Wyeth/ViroPharma), IDX-184 (Idenix), IDX-375 (Idenix), NM-283 (Idenix/Novartis), GL-60667 (Genelabs), JTK-109 (Japan Tobacco), PSI-6130 (Pharmasset), R1479 (Roche), R-1626 (Roche), R-7128 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH-222/VX-222 (ViroChem/Vertex), VCH-916 (ViroChem), VCH-716 (ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004).

Other HCV polymerase inhibitors useful in the present compositions and methods include, but are not limited to, those disclosed in International Publication Nos. WO 08/082484, WO 08/082488, WO 08/083351, WO 08/136815, WO 09/032116, WO 09/032123, WO 09/032124 and WO 09/032125; and the following compounds:

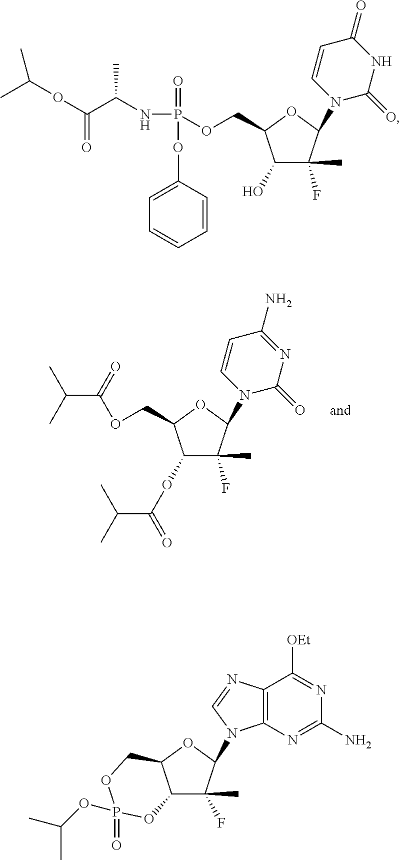

and pharmaceutically acceptable salts thereof

Interferons useful in the present compositions and methods include, but are not limited to, interferon alfa-2a, interferon alfa-2b, interferon alfacon-1 and petroleum etherG-interferon alpha conjugates. “PEG-interferon alpha conjugates” are interferon alpha molecules covalently attached to a petroleum etherG molecule. Illustrative petroleum etherG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name petroleum etherG-Intron™ from Schering-Plough Corporation), interferon alpha-2b-XL (e.g., as sold under the trade name petroleum etherG-Intron™), interferon alpha-2c (Berofor Alpha™, Boehringer Ingelheim, Ingelheim, Germany), petroleum etherG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), R-7025 (Roche), IFN-α-2b-XL (Flamel Technologies), belerofon (Nautilus) and consensus interferon as defined by determination of a consensus sequence of naturally occurring interferon alphas (Infergen™, Amgen, Thousand Oaks, Calif.).

Examples of viral protease inhibitors useful in the present compositions and methods include, but are not limited to, an HCV protease inhibitor. Examples of HCV protease inhibitors useful in the present compositions and methods include, but are not limited to, VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott/Enanta), TMC-435350 (Medivir), RG7227 (Danoprevir, InterMune/Roche), EA-058 (Abbott/Enanta), EA-063 (Abbott/Enanta), GS-9256 (Gilead), IDX-320 (Idenix), ACH-1625 (Achillion), ACH-2684 (Achillion), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhibitors useful in the present compositions and methods include, but are not limited to, the following compounds:

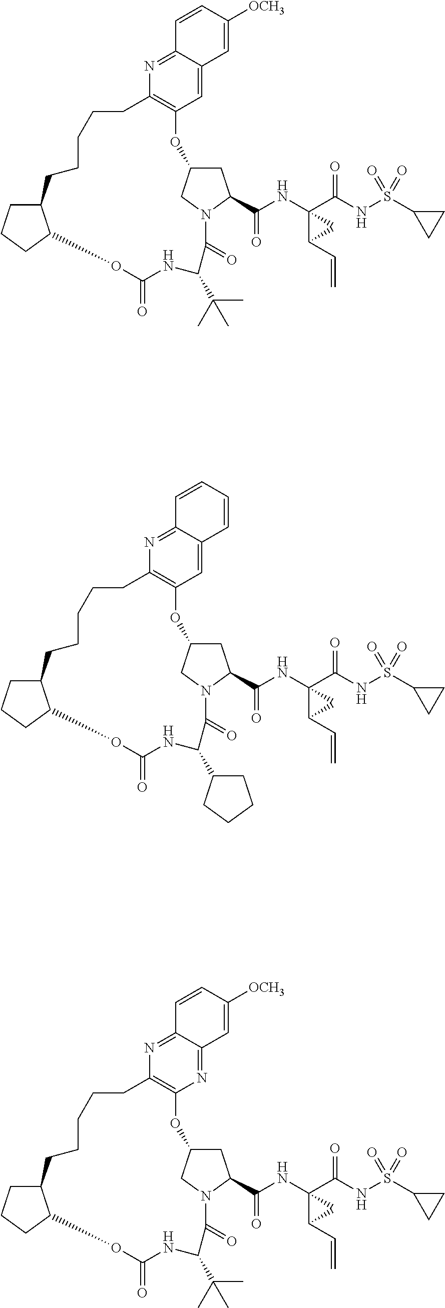

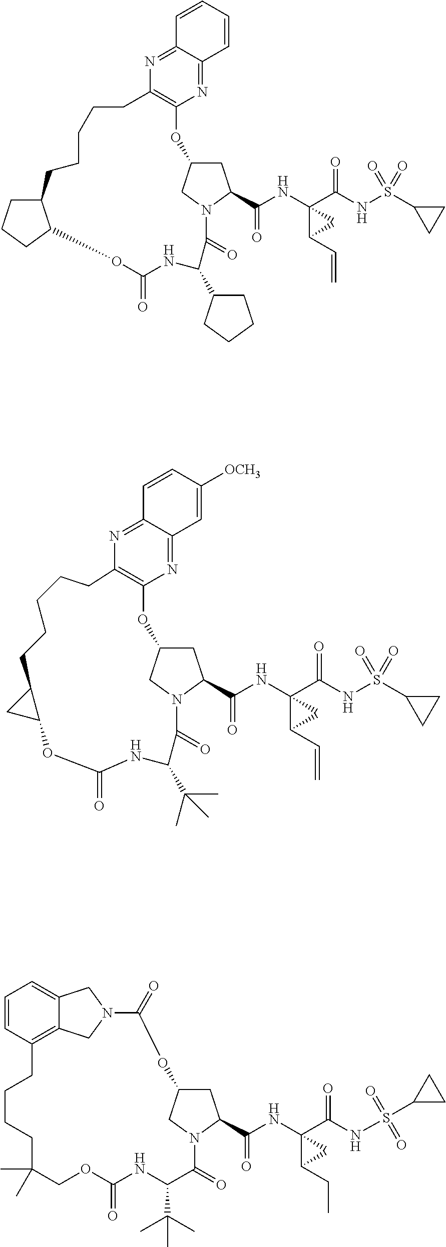

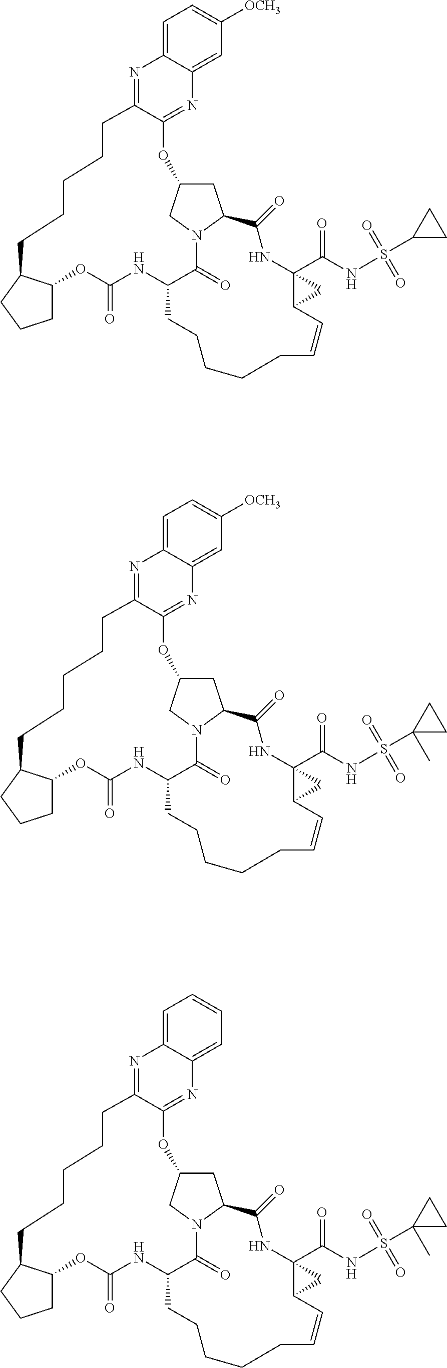 [SMILES: COc1ccc2nc3c(nc2c1)O[C@@H]1C[C@H]2C(=O)N[C@]4(C(=O)NS(=O)(=O)C5CC5)C[C@H]4/C=C\CCCCC[C@H](NC(=O)O[C@@H]4CCC[C@H]4CCCCC3)C(=O)N2C1 ; COc1ccc2nc3c(nc2c1)O[C@@H]1C[C@H]2C(=O)N[C@]4(C(=O)NS(=O)(=O)C5(C)CC5)C[C@H]4/C=C\CCCCC[C@H](NC(=O)O[C@@H]4CCC[C@H]4CCCCC3)C(=O)N2C1 ; CC1(S(=O)(=O)NC(=O)[C@@]23C[C@H]2/C=C\CCCCC[C@@H]2NC(=O)O[C@@H]4CCC[C@H]4CCCCCc4nc5ccccc5nc4O[C@@H]4C[C@@H](C(=O)N3)N(C4)C2=O)CC1]

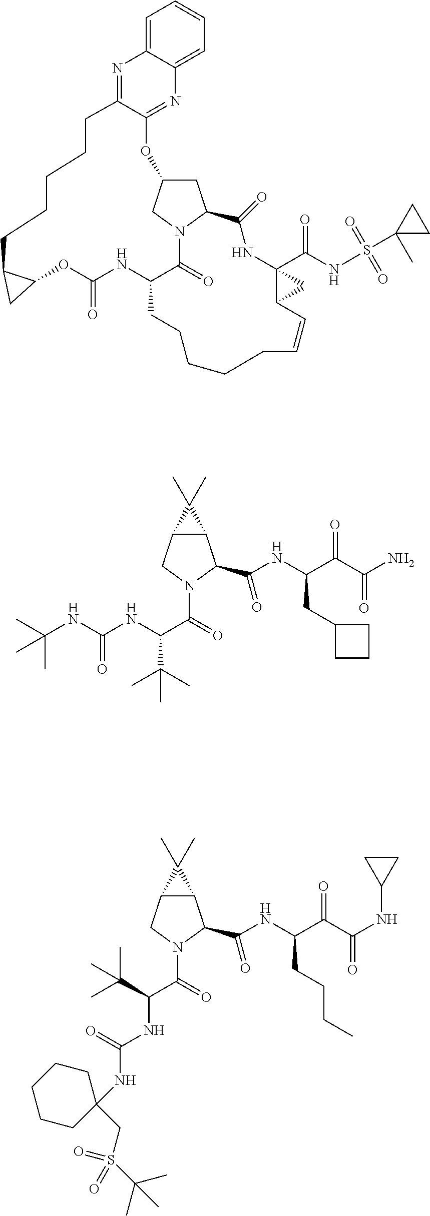 [SMILES: CC1(S(=O)(=O)NC(=O)[C@@]23C[C@H]2/C=C\CCCCC[C@@H]2NC(=O)O[C@@H]4C[C@H]4CCCCCc4nc5ccccc5nc4O[C@@H]4C[C@@H](C(=O)N3)N(C4)C2=O)CC1 ; CC(C)(C)NC(=O)N[C@H](C(=O)N1C[C@H]2[C@@H]([C@H]1C(=O)N[C@H](CC1CCC1)C(=O)C(N)=O)C2(C)C)C(C)(C)C ; CCCC[C@@H](NC(=O)[C@@H]1[C@@H]2[C@H](CN1C(=O)[C@@H](NC(=O)NC1(CS(=O)(=O)C(C)(C)C)CCCCC1)C(C)(C)C)C2(C)C)C(=O)C(=O)NC1CC1]

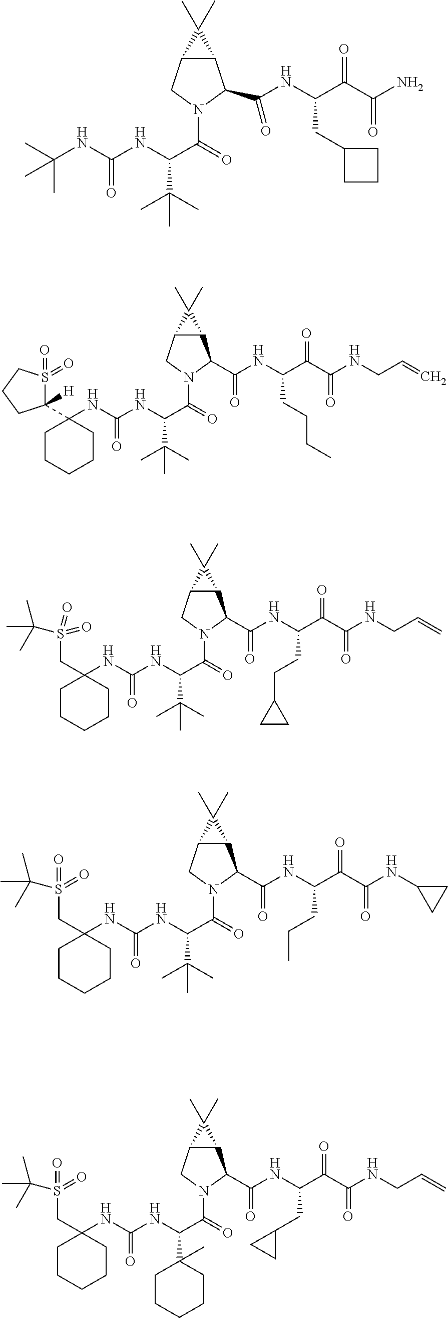

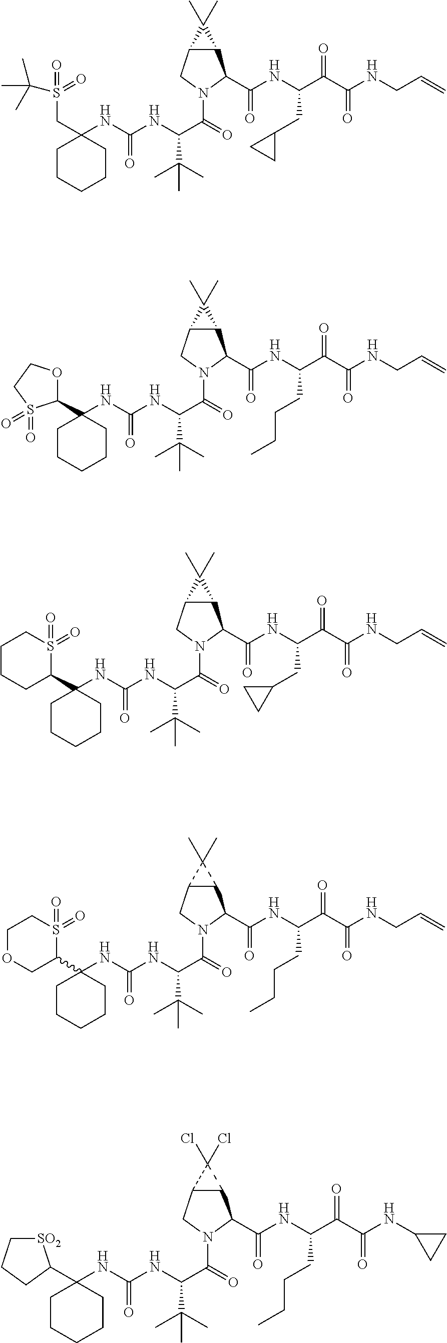

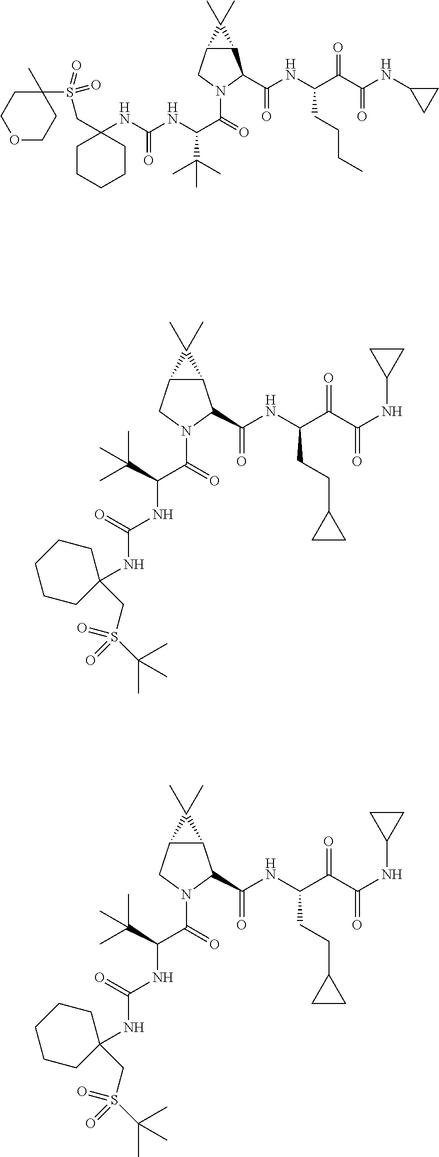

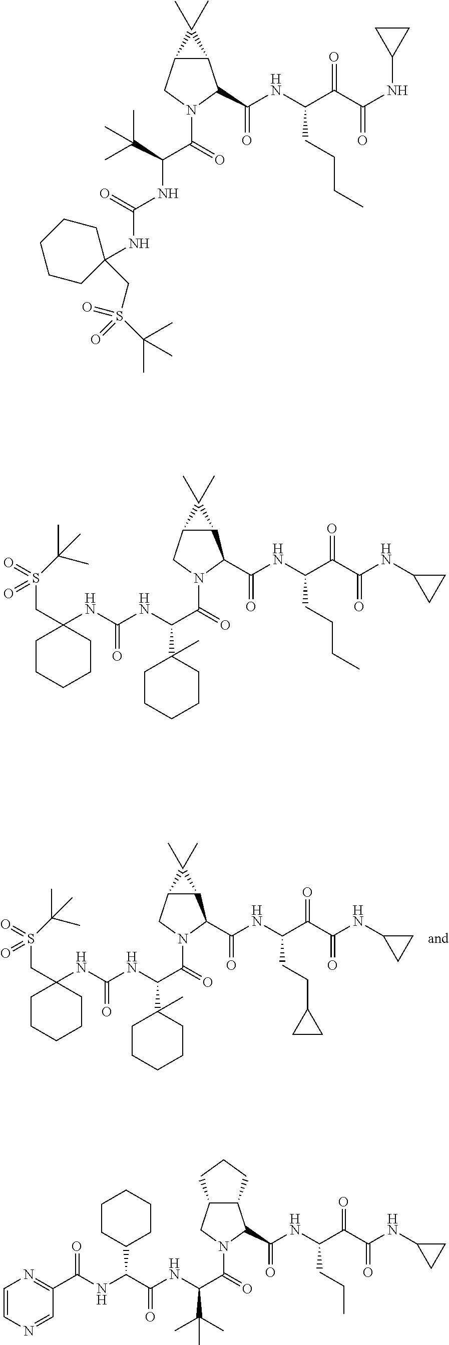

and pharmaceutically acceptable salts thereof

Viral replication inhibitors useful in the present compositions and methods include, but are not limited to, HCV replicase inhibitors, IRES inhibitors, NS4A inhibitors, NS3 helicase inhibitors, NS5A inhibitors, NS5B inhibitors, ribavirin, AZD-2836 (Astra Zeneca), viramidine, A-831 (Arrow Therapeutics), EDP-239 (Enanta), ACH-2928 (Achillion), GS-5885 (Gilead); an antisense agent or a therapeutic vaccine.

HCV NS5A inhibitors useful in the present compositions and methods include, but are not limited to, ACH-2928 (Achilon), A-832 (Arrow Therpeutics), AZD-7295 (Astra Zeneca/Arrow), GS-5885 (Gilead), PPI-461 (Presidio), PPI-1301 (Presidio), BMS-824383 (Bristol-Myers Squibb) and BMS-790052 (Bristol-Myers Squibb). Additional HCV NS5A inhibitors useful as second additional therapeutic agents in the present compositions and methods include, but are not limited to those disclosed in International Publication No. WO 2010/111483 and the following compounds:

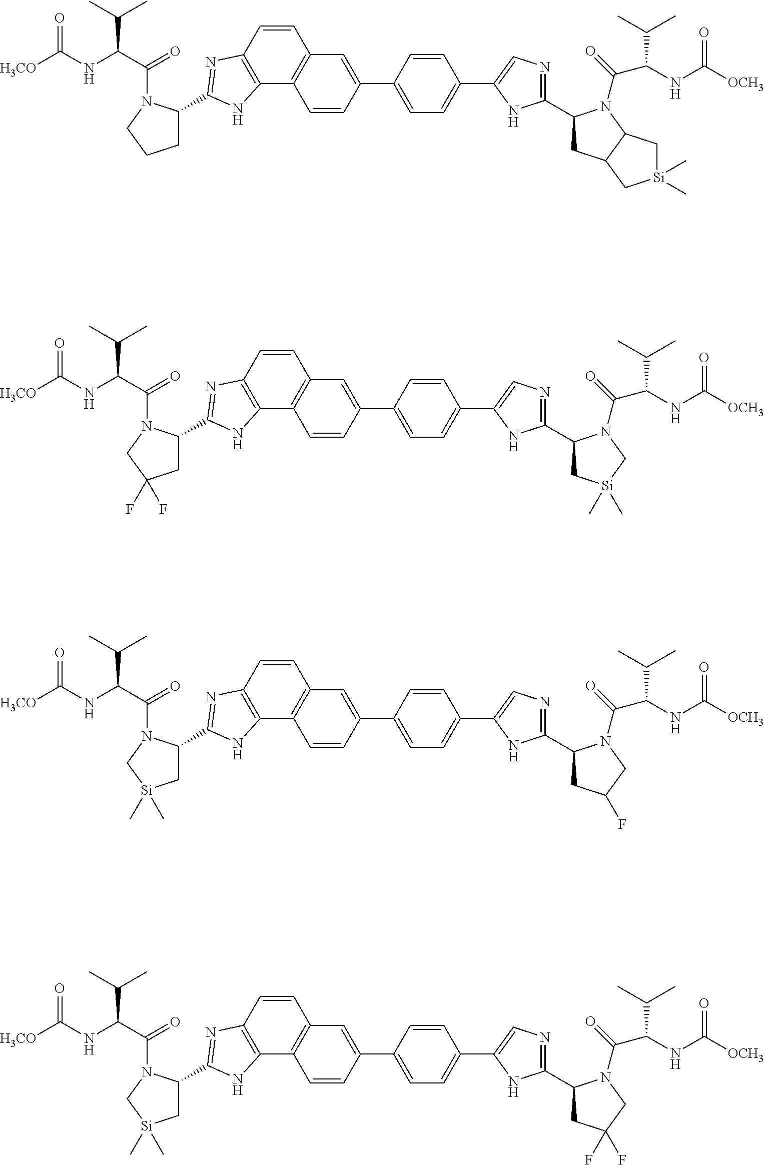

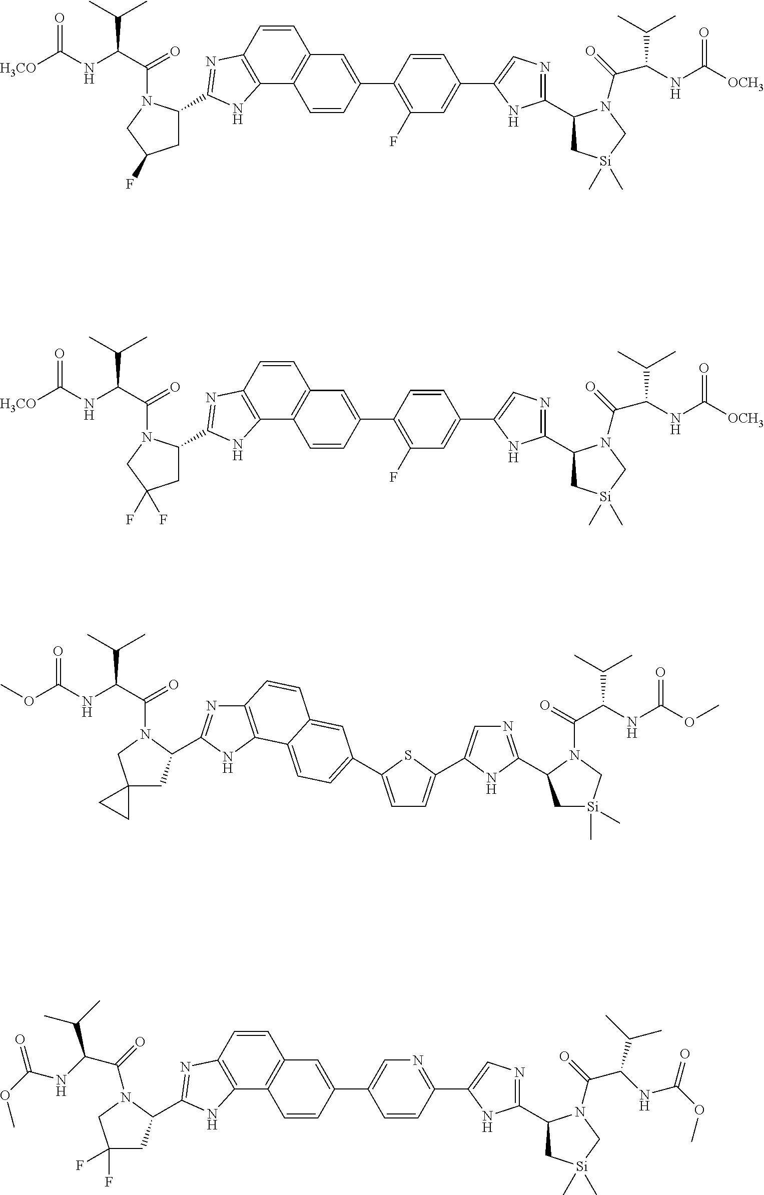

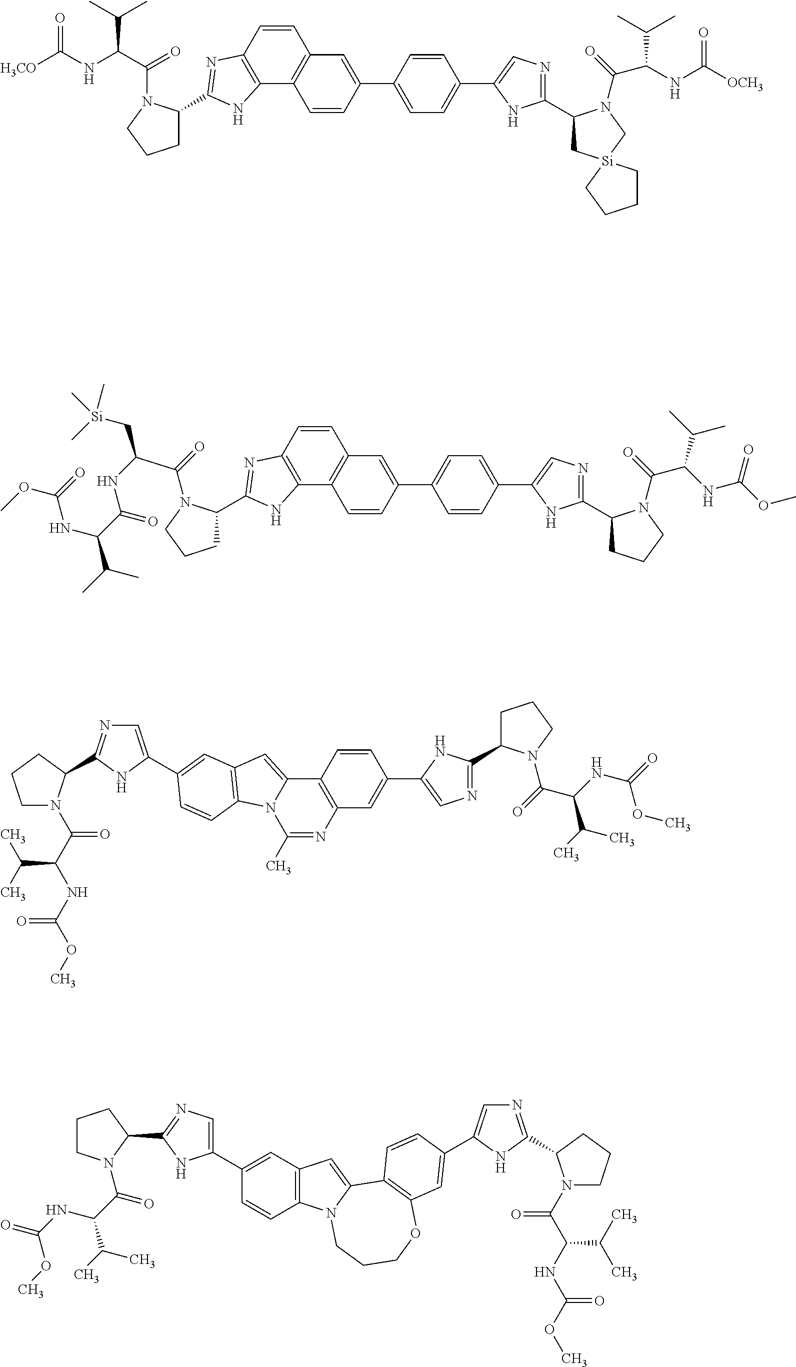

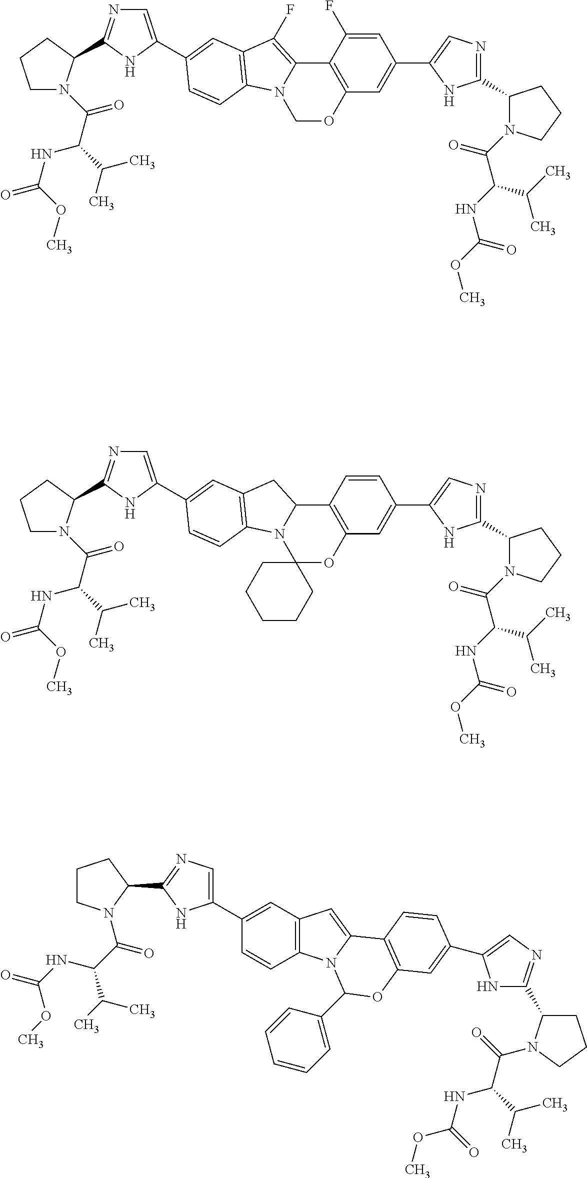 [SMILES: COC(=O)N[C@H](C(=O)N1CCC[C@H]1c1ncc(-c2cc(F)c3c(c2)OCn2c-3c(F)c3cc(-c4cnc([C@@H]5CCCN5C(=O)[C@@H](NC(=O)OC)C(C)C)[nH]4)ccc32)[nH]1)C(C)C ; COC(=O)N[C@H](C(=O)N1CCC[C@H]1c1ncc(-c2ccc3c(c2)CC2c4ccc(-c5cnc([C@@H]6CCCN6C(=O)[C@@H](NC(=O)OC)C(C)C)[nH]5)cc4OC4(CCCCC4)N32)[nH]1)C(C)C ; COC(=O)N[C@H](C(=O)N1CCC[C@H]1c1ncc(-c2ccc3c(c2)OC(c2ccccc2)n2c-3cc3cc(-c4cnc([C@@H]5CCCN5C(=O)[C@@H](NC(=O)OC)C(C)C)[nH]4)ccc32)[nH]1)C(C)C]

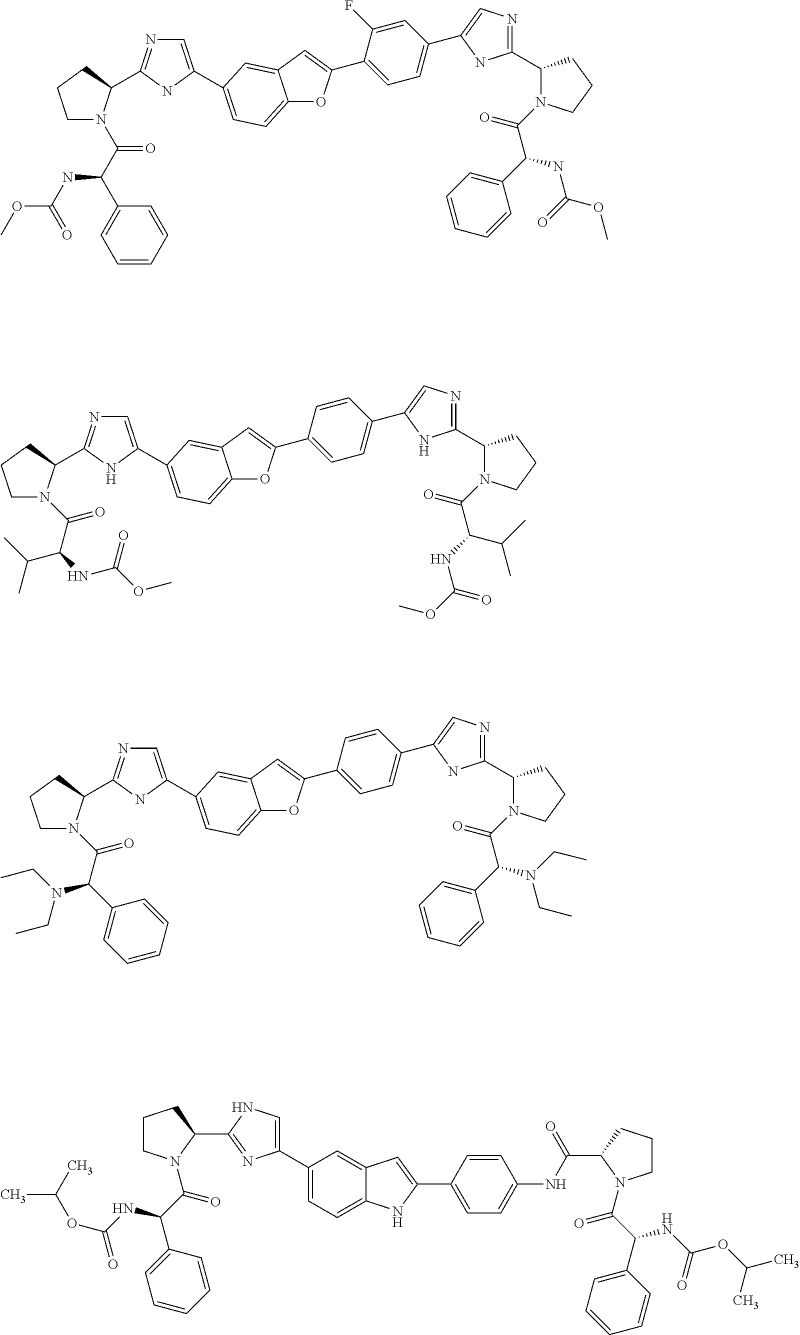

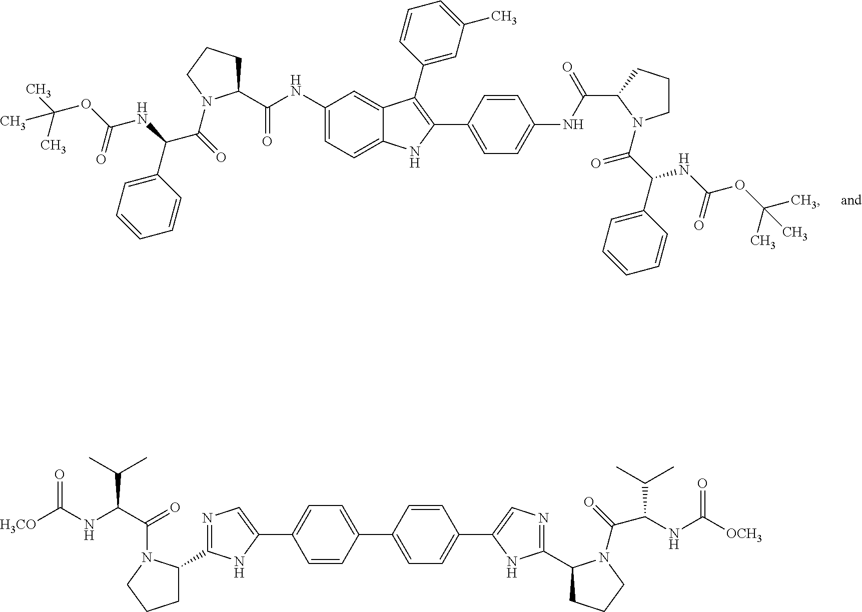 [SMILES: Cc1cccc(-c2c(-c3ccc(NC(=O)[C@@H]4CCCN4C(=O)[C@H](NC(=O)OC(C)(C)C)c4ccccc4)cc3)[nH]c3ccc(NC(=O)[C@@H]4CCCN4C(=O)[C@H](NC(=O)OC(C)(C)C)c4ccccc4)cc23)c1 ; COC(=O)N[C@H](C(=O)N1CCC[C@H]1c1ncc(-c2ccc(-c3ccc(-c4cnc([C@@H]5CCCN5C(=O)[C@@H](NC(=O)OC)C(C)C)[nH]4)cc3)cc2)[nH]1)C(C)C]

and pharmaceutically acceptable salts thereof

HCV replicase inhibitors useful in the present compositions and methods include, but are not limited to, those disclosed in U.S. Patent Publication No. US20090081636.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). A compound of the invention and an additional therapeutic agent may be present in fixed amounts (dosage amounts) in a single dosage unit (e.g., a capsule, a tablet and the like).

The HCV NS5B inhibitory activity of the present compounds may be tested using assays known in the art. The HCV NS5B polymerase inhibitors described herein have activities in a genotype 1b replicon assay as described in the Examples. The assay is performed by incubating a replicon harboring cell-line in the presence of inhibitor for a set period of time and measuring the effect of the inhibitor on HCV replicon replication either directly by quantifying replicon RNA level, or indirectly by measuring enzymatic activity of a co-encoded reporter enzyme such as luciferase or β-lactamase. By performing a series of such measurements at different inhibitor concentrations, the effective inhibitory concentration of the inhibitor (EC₅₀ or EC₉₀) is determined. See Jan M. Vrolijk et al., A replicons-based bioassay for the measurement of interferons in patients with chronic hepatitis C, 110 J. VIROLOGICAL METHODS 201 (2003). Such assays may also be run in an automated format for high through-put screening. See Paul Zuck et al., A cell-based β-lactamase reporter gene assay for the identification of inhibitors of hepatitis C virus replication, 334 ANALYTICAL BIOCHEMISTRY 344 (2004).

The present invention also includes processes for making compounds of formula I. The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above. The following reaction schemes and examples serve only to illustrate the invention and its practice.

### General Schemes

The compounds of Formula (I) may be prepared from known or readily prepared starting materials, following methods known to one skilled in the art of organic synthesis. Methods useful for making the compounds of Formula (I) are set forth in the Examples below and generalized in Schemes 1-2 below. Alternative synthetic pathways and analogous structures will be apparent to those skilled in the art of organic synthesis. All stereoisomers and tautomeric forms of the compounds are contemplated.

Some commercially available starting materials and intermediates used for the synthesis of the compounds of Formula (I) are available. These starting materials and intermediates are available from commercial suppliers such as Sigma-Aldrich (St. Louis, Mo.) and Acros Organics Co. (Fair Lawn, N.J.). Such starting materials and intermediates compounds are used as received.

Scheme 1 shows methods useful for making formula K.

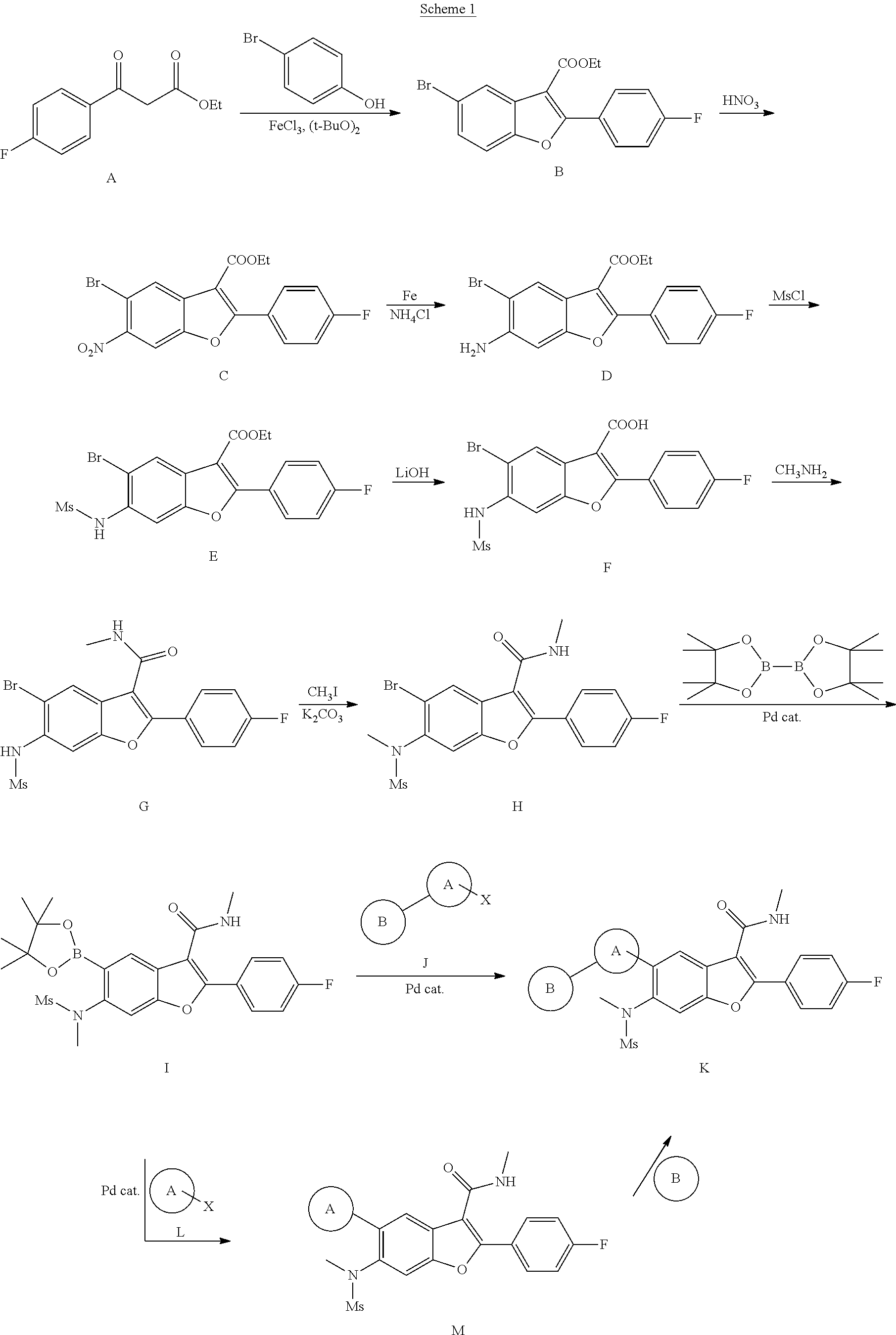

Commercially available compound A can be cyclized with 4-bromophenol to provide benzofuran compound B. Nitration of compound B provides nitrocompound C, which can be reduced to provide amine compound D. Mesylation of the amino group of D provides compound E, which can then be hydrolyzed using LiOH, for example, to provide the carboxylic acid compound F. The carboxylic acid of compound F is then condensed with methanamine using common amide forming reagents such as EDCI and HOBT to provide compound G. The sulfonamide group of G can be coupled with MeI in the presence of potassium carbonate to provide compound H. Compound H can be converted to corresponding boronic ester I using bis(pinacolato)diboron in the presence of a palladium catalyst. Finally, compound I can be reacted with substituted bicyclic heteroaryl halides of formula J to provide the compounds of formula K. Alternatively, compound I can be reacted with substituted heteroaryl halides of formula L to provide the compounds of formula M, which will be further functionalized to afford compound K.

Scheme 2 shows an alternate method useful for making compounds of formula S.

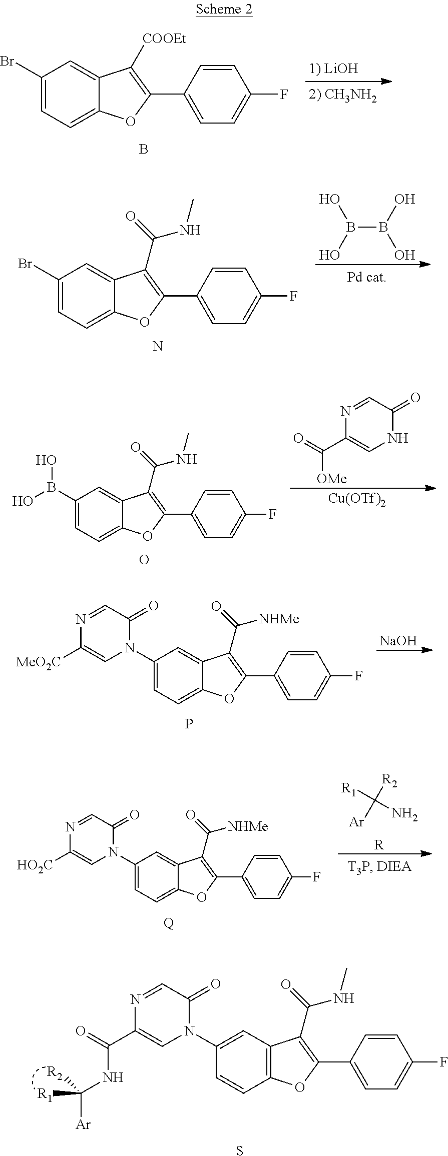

### LIST OF ABBREVIATIONS

### a.q., aq Aqueous

Boc t-butyloxycarbonyl
(t-BuO)₂ Di-tert-butyl peroxide
cat. Catalyst

### Cbz Carbobenzyloxy

Cs₂CO₃ Cesium carbonate
Cu(OTf)₂ Copper(II) triflate
DCM, CH₂Cl₂ Dichloromethane

### DIEA N, N-Diisopropylethylamine

### DMF Dimethylformamide

### DMSO Dimethylsulfoxide

EDCI N-(3-Dimethylaminopropyl)-N′-ethylcarbodiimide (also EDC)

### Et Ethyl

EtOAc, EA Ethyl acetate

### Fe Iron

FeCl₃ Iron chloride
HCl Hydrochloric acid
HNO₃ Nitric acid

### H₂O Water

HOBT 1-Hydroxy benzotriazole

### HPLC High Performance Liquid Chromatography

K₂CO₃ Potassium carbonate
K₃PO₄ Potassium Phosphate
LDA Lithium diisopropylamide
LiOH Lithium hydroxide

### Me Methyl

MeNH₂, CH₃NH₂ Methanamine
Met, CH₃I Methyl iodide

### MeOH, CH₃OH Methanol

MS Mass spectroscopy
Ms Methanesulfonyl (or mesyl) group
MsCl Methanesulfonyl chloride
N₂ Nitrogen gas or atmosphere
NaHCO₃ Sodium bicarbonate
Nat Sodium iodide
NaNO₂ Sodium nitrite
NaOH Sodium hydroxide
NaOMe Sodium methoxide
Na₂SO₄ Sodium sulfate (anhydrous)

### NBS N-Bromosuccinimide

NH₄Cl Ammonium chloride

### NIS N-iodosuccinimide

### Pd Palladium

Pd₂(dba)₃ Tris(dibenzylideneacetone)dipalladium(0)
Pd(dppf)Cl₂ 1,1′-bis(diphenylphosphino)ferrocene-palladium(II)dichloride
Pd(PPh₃)₂Cl₂ Bis(triphenylphosphine)palladium(II) dichloride
RT Room temperature, approximately 25° C.

### T3P Propylphosphonic Anhydride

### Tos Tosyl

TFA Trifluoroacetic acid

### THF Tetrahydrofuran

TLC Thin layer chromatography
ZnCl₂ Zinc chloride

### EXAMPLES

### Example 1

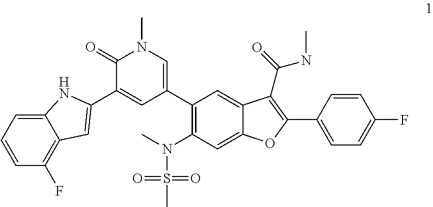 [SMILES: CNC(=O)c1c(-c2ccc(F)cc2)oc2cc(N(C)S(C)(=O)=O)c(-c3cc(-c4cc5c(F)cccc5[nH]4)c(=O)n(C)c3)cc12]

### Step 1—Synthesis of 2-(4-fluorophenyl)-N-methyl-5-(1-methyl-6-oxo-1,6-dihydropyridin-3-yl)-6-(N-methylmethylsulfonamido)benzofuran-3-carboxamide

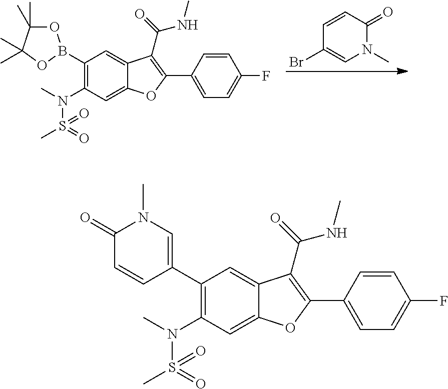

To a degassed solution of 2-(4-fluorophenyl)-N-methyl-6-(N-methylmethylsulfonamido)-5-(4,4,5,5-tetramethyl-1,3,2-dioxaborolan-2-yl)benzofuran-3-carboxamide (500 mg, 1.0 mmol) and 5-bromo-1-methylpyridin-2(1H)-one (281 mg, 1.5 mmol) in 1,4-dioxane (8 mL) and water (200 μl) was added CS₂CO₃ (486 mg, 1.5 mmol) and 1,1′-bis(di-tert-butylphosphino)ferrocene palladium dichloride (32 mg, 0.05 mmol) under N₂ protection. The resulting mixture was heated to 80° C. and stirred at this temperature overnight. The reaction was cooled, filtered through a pad of the celite and washed with ethyl acetate. The combined filtrate was evaporated in vacuo. The resulting residue was purified using column chromatography (eluted with 0-100% EtOAc/hexane) to provide 2-(4-fluorophenyl)-N-methyl-5-(1-methyl-6-oxo-1,6-dihydropyridin-3-yl)-6-(N-methylmethylsulfonamido)benzofuran-3-carboxamide (350 mg, yield: 73%). MS (M+H)⁺: 484.

### Step 2—Synthesis of 5-(5-bromo-1-methyl-6-oxo-1,6-dihydropyridin-3-yl)-2-(4-fluorophenyl)-N-methyl-6-(N-methylmethylsulfonamido)benzofuran-3-carboxamide

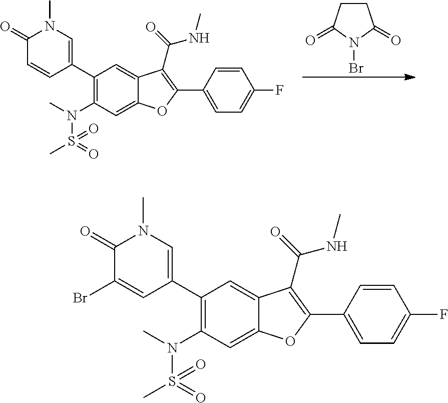 [SMILES: CNC(=O)c1c(-c2ccc(F)cc2)oc2cc(N(C)S(C)(=O)=O)c(-c3ccc(=O)n(C)c3)cc12 ; O=C1CCC(=O)N1Br ; CNC(=O)c1c(-c2ccc(F)cc2)oc2cc(N(C)S(C)(=O)=O)c(-c3cc(Br)c(=O)n(C)c3)cc12]

To a screw cap vial was added 2-(4-fluorophenyl)-N-methyl-5-(1-methyl-6-oxo-1,6-dihydropyridin-3-yl)-6-(N-methylmethylsulfonamido)benzofuran-3-carboxamide (300 mg, 0.62 mmol) and NBS (221 mg, 1.24 mmol) in acetonitrile (5 ml). The vial was capped and microwaved at 80° C. for 20 min. The reaction mixture was evaporated in vacuo to remove the volatiles. The resulting residue was purified by column chromatography (eluted with 0-4% MeOH/DCM) to provide 5-(5-bromo-1-methyl-6-oxo-1,6-dihydropyridin-3-yl)-2-(4-fluorophenyl)-N-methyl-6-(N-methylmethylsulfonamido)benzofuran-3-carboxamide (290 mg, yield 83%). MS (M+H)⁺: 564.

### Step 3—Synthesis of compound 5-(5-(4-fluoro-1H-indol-2-yl)-1-methyl-6-oxo-1,6-dihydropyridin-3-yl)-2-(4-fluorophenyl)-N-methyl-6-(N-methylmethylsulfonamido)benzofuran-3-carboxamide

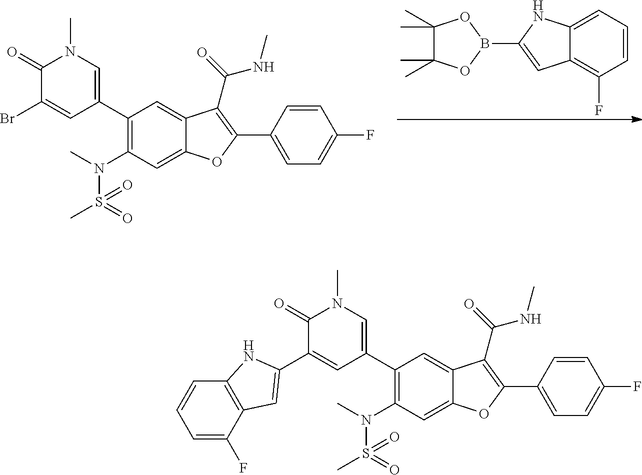 [SMILES: CNC(=O)c1c(-c2ccc(F)cc2)oc2cc(N(C)S(C)(=O)=O)c(-c3cc(Br)c(=O)n(C)c3)cc12 ; CC1(C)OB(c2cc3c(F)cccc3[nH]2)OC1(C)C ; CNC(=O)c1c(-c2ccc(F)cc2)oc2cc(N(C)S(C)(=O)=O)c(-c3cc(-c4cc5c(F)cccc5[nH]4)c(=O)n(C)c3)cc12]

To a solution of 5-(5-bromo-1-methyl-6-oxo-1,6-dihydropyridin-3-yl)-2-(4-fluorophenyl)-N-methyl-6-(N-methylmethylsulfonamido)benzofuran-3-carboxamide (60 mg, 0.11 mmol) and 4-fluoro-2-(4,4,5,5-tetramethyl-1,3,2-dioxaborolan-2-yl)-1H-indole (56 mg, 0.21 mmol) in 1,4-dioxane (1.5 mL) and water (100 μl) was added CS₂CO₃ (70 mg, 0.21 mmol) and 1,1′-bis(di-tert-butylphosphino)ferrocene palladium dichloride (10 mg, 0.02 mmol) under N₂ protection. The resulting mixture was heated to 65° C. and stirred at this temperature overnight. The reaction was cooled, filtered through a pad of the celite and washed with ethyl acetate. The combined filtrate was evaporated in vacuo. The resulting residue was purified using preparative TLC (eluted with 4% MeOH/DCM) to provide 5-(5-(4-fluoro-1H-indol-2-yl)-1-methyl-6-oxo-1,6-dihydropyridin-3-yl)-2-(4-fluorophenyl)-N-methyl-6-(N-methylmethylsulfonamido)benzofuran-3-carboxamide (23 mg, yield: 35%). MS (M+H)⁺: 617.

Examples 2-4, depicted in the table below, were prepared using the method described above.

| | | | MS |
|---|---|---|---|
| Example | Structure | IUPAC Name | (M + H)⁺ |
| 2 | 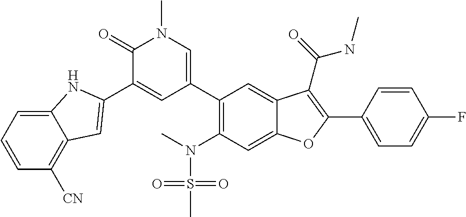 [SMILES: CNC(=O)c1c(-c2ccc(F)cc2)oc2cc(N(C)S(C)(=O)=O)c(-c3cc(-c4cc5c(C#N)cccc5[nH]4)c(=O)n(C)c3)cc12] | 5-(5-(4-cyano-1H-indol-2-yl)- 1-methyl-6-oxo-1,6- dihydropyridin-3-yl)-2-(4- fluorophenyl)-N-methyl-6-(N- methylmethylsulfonamido)- benzofuran-3-carboxamide | 624 |
| 3 | 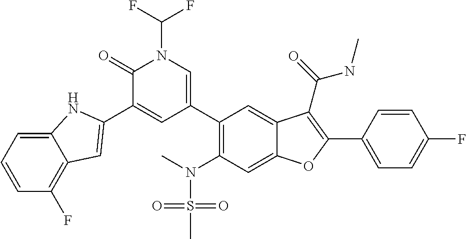 [SMILES: CNC(=O)c1c(-c2ccc(F)cc2)oc2cc(N(C)S(C)(=O)=O)c(-c3cc(-c4cc5c(F)cccc5[nH]4)c(=O)n(C(F)F)c3)cc12] | 5-(1-(difluoromethyl)-5-(4- fluoro-1H-indol-2-yl)-6-oxo- 1,6-dihydropyridin-3-yl)-2-(4- fluorophenyl)-N-methyl- 6-(N-methylmethyl- sulfonamido)benzofuran- 3-carboxamide | 653 |
| 4 | 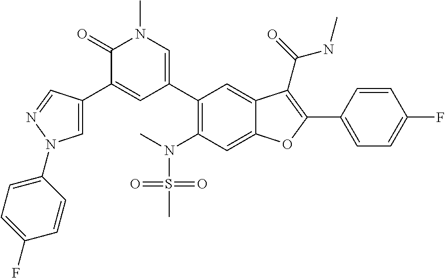 [SMILES: CNC(=O)c1c(-c2ccc(F)cc2)oc2cc(N(C)S(C)(=O)=O)c(-c3cc(-c4cnn(-c5ccc(F)cc5)c4)c(=O)n(C)c3)cc12] | 2-(4-fluorophenyl)-5-(5-(1-(4- fluorophenyl)-1H-pyrazol-4- yl)-1-methyl-6-oxo-1,6- dihydropyridin-3-yl)-N- methyl-6-(N-methyl- methylsulfonamido)benzo- furan-3-carboxamide | 644 |

### Example 5

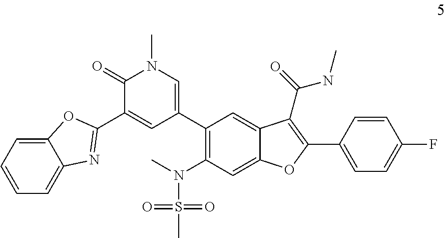 [SMILES: CNC(=O)c1c(-c2ccc(F)cc2)oc2cc(N(C)S(C)(=O)=O)c(-c3cc(-c4nc5ccccc5o4)c(=O)n(C)c3)cc12]

### Step 1—Synthesis of 2-(4-fluorophenyl)-5-(5-iodo-1-methyl-6-oxo-1,6-dihydropyridin-3-yl)-N-methyl-6-(N-methylmethylsulfonamido)benzofuran-3-carboxamide

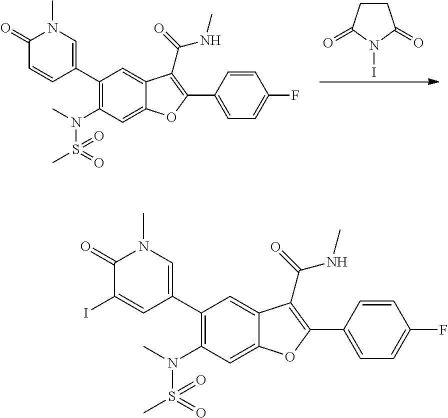 [SMILES: CNC(=O)c1c(-c2ccc(F)cc2)oc2cc(N(C)S(C)(=O)=O)c(-c3ccc(=O)n(C)c3)cc12 ; O=C1CCC(=O)N1I ; CNC(=O)c1c(-c2ccc(F)cc2)oc2cc(N(C)S(C)(=O)=O)c(-c3cc(I)c(=O)n(C)c3)cc12]

To a screw cap vial was added 2-(4-fluorophenyl)-N-methyl-5-(1-methyl-6-oxo-1,6-dihydropyridin-3-yl)-6-(N-methylmethylsulfonamido)benzofuran-3-carboxamide (450 mg, 0.93 mmol) and NIS (419 mg, 1.86 mmol) in acetonitrile (5 ml). The vial was capped and microwaved at 80° C. for 20 min. The reaction mixture was evaporated in vacuo to remove the volatiles. The resulting residue was purified by column chromatography (eluted with 0-3% MeOH/DCM) to provide 2-(4-fluorophenyl)-5-(5-iodo-1-methyl-6-oxo-1,6-dihydropyridin-3-yl)-N-methyl-6-(N-methylmethylsulfonamido)benzofuran-3-carboxamide (290 mg, yield 51%). MS (M+H)⁺:610.

### Step 2—Synthesis of compound 5-(5-(benzo[d]oxazol-2-yl)-1-methyl-6-oxo-1,6-dihydropyridin-3-yl)-2-(4-fluorophenyl)-N-methyl-6-(N-methylmethylsulfonamido)benzofuran-3-carboxamide

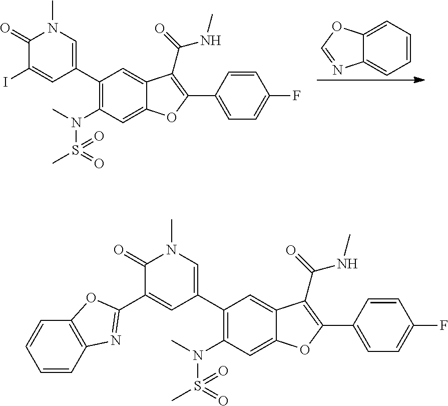 [SMILES: CNC(=O)c1c(-c2ccc(F)cc2)oc2cc(N(C)S(C)(=O)=O)c(-c3cc(I)c(=O)n(C)c3)cc12 ; c1ccc2ocnc2c1 ; CNC(=O)c1c(-c2ccc(F)cc2)oc2cc(N(C)S(C)(=O)=O)c(-c3cc(-c4nc5ccccc5o4)c(=O)n(C)c3)cc12]

To a solution of benzo[d]oxazole (50 mg, 0.41 mmol) in THF (2 mL) at 0° C. was added 2,2,6,6-Bis(tetramethylpiperidine)zinc, lithium chloride complex 0.35 M in toluene (1.4 ml, 0.41 mmol) and stirred under N₂ protection for 2 h, then followed by addition of 2-(4-fluorophenyl)-5-(5-iodo-1-methyl-6-oxo-1,6-dihydropyridin-3-yl)-N-methyl-6-(N-methylmethylsulfonamido)benzofuran-3-carboxamide (50 mg, 0.08 mmol) and chloro(2-dicyclohexylphosphino-2′,6′-dimethoxy-1,1′-biphenyl)[2-(2′-amino-1,1′-biphenyl)]palladium(II) (11 mg, 0.02 mmol). The resulting mixture was stirred at RT for 2 h, then heated to 50° C. and stirred for additional 3 h. The mixture was cooled, diluted with ethyl acetate, washed with saturated NH₄Cl and brine, dried (Na₂SO₄), filtered and the solvent was evaporated under reduced pressure. The residue was purified by preparative TLC eluting with 3% MeOH/DCM to give 5-(5-(benzo[d]oxazol-2-yl)-1-methyl-6-oxo-1,6-dihydropyridin-3-yl)-2-(4-fluorophenyl)-N-methyl-6-(N-methylmethylsulfonamido)benzofuran-3-carboxamide (35 mg, yield 71%). MS (M+H)⁺: 601.

### Example 6

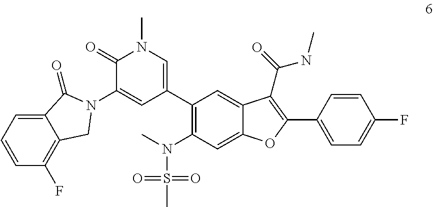 [SMILES: CNC(=O)c1c(-c2ccc(F)cc2)oc2cc(N(C)S(C)(=O)=O)c(-c3cc(N4Cc5c(F)cccc5C4=O)c(=O)n(C)c3)cc12]

5-(5-bromo-1-methyl-6-oxo-1,6-dihydropyridin-3-yl)-2-(4-fluorophenyl)-N-methyl-6-(N-methylmethylsulfonamido)benzofuran-3-carboxamide (22 mg, 0.04 mmol), 4-fluoroisoindolin-1-one, Cs₂CO₃ (26 mg, 0.08 mmol) and Xantphos precatalyst (7 mg, 0.007 mmol) were combined with 1,4-dioxane (1.0 ml) in a sealed tube. The resulting mixture was heated to 90° C. and stirred for 2 h, then heated to 110° C. and stirred for additional 2 h. The filtration through a pad of the celite removed the solid. After washed with ethyl acetate, the combined filtrate was evaporated in vacuo. The resulting residue was purified by column chromatography (eluted with 0-5% MeOH/DCM) to provide 5-(5-(4-fluoro-1-oxoisoindolin-2-yl)-1-methyl-6-oxo-1,6-dihydropyridin-3-yl)-2-(4-fluorophenyl)-N-methyl-6-(N-methylmethylsulfonamido)benzofuran-3-carboxamide (13 mg, yield: 53%). MS (M+H)⁺: 633.

### Example 7

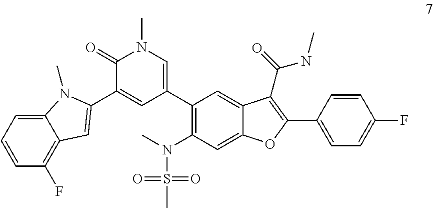 [SMILES: CNC(=O)c1c(-c2ccc(F)cc2)oc2cc(N(C)S(C)(=O)=O)c(-c3cc(-c4cc5c(F)cccc5n4C)c(=O)n(C)c3)cc12]

5-(5-(4-Fluoro-1H-indol-2-yl)-1-methyl-6-oxo-1,6-dihydropyridin-3-yl)-2-(4-fluorophenyl)-N-methyl-6-(N-methylmethylsulfonamido)benzofuran-3-carboxamide (15 mg, 0.02 mmol), MeI (35 mg, 0.24 mmol) and Cs₂CO₃ (40 mg, 0.12 mmol) were combined in DMF (1 ml). The mixture was stirred at RT for 1 h. Filtration through a celite pad removed the solid. After washing with ethyl acetate, the combined filtrate was concentrated in vacuo. The resulting residue was purified by preparative TLC (eluted with ethyl acetate) to provide 5-(5-(4-fluoro-1-methyl-1H-indol-2-yl)-1-methyl-6-oxo-1,6-dihydropyridin-3-yl)-2-(4-fluorophenyl)-N-methyl-6-(N-methylmethylsulfonamido)benzofuran-3-carboxamide (8 mg, yield: 52%). MS (M+H)⁺: 631.

Example 8, depicted in the table below, was prepared using the method described above.

| | | | MS |
|---|---|---|---|
| Example | Structure | IUPAC Name | (M + H)⁺ |
| 8 | 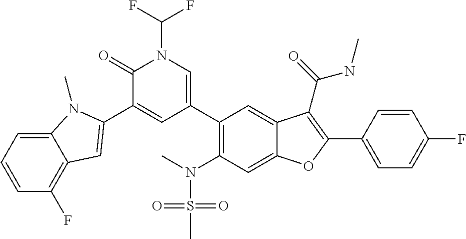 [SMILES: CNC(=O)c1c(-c2ccc(F)cc2)oc2cc(N(C)S(C)(=O)=O)c(-c3cc(-c4cc5c(F)cccc5n4C)c(=O)n(C(F)F)c3)cc12] | 5-(1-(difluoromethyl)-5-(4- fluoro-1-methyl-1H-indol-2- yl)-6-oxo-1,6-dihydropyridin- 3-yl)-2-(4-fluorophenyl)-N- methyl-6-(N-methyl- methylsulfonamido)benzo- furan-3-carboxamide | 667 |

### Example 9

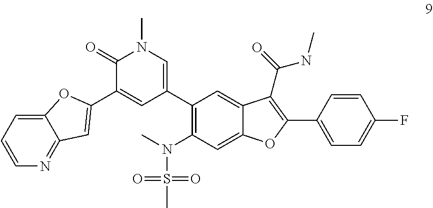 [SMILES: CNC(=O)c1c(-c2ccc(F)cc2)oc2cc(N(C)S(C)(=O)=O)c(-c3cc(-c4cc5ncccc5o4)c(=O)n(C)c3)cc12]

### Step 1—Synthesis of 5-bromo-3-(furo[3,2-b]pyridin-2-yl)-1-methylpyridin-2(1H)-one

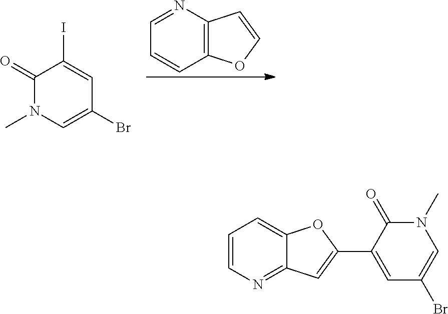

To a solution of furo[3,2-b]pyridine (100 mg, 0.32 mmol) in THF (2 mL) at −78° C. was added 2.0 M LDA in THF (0.24 ml, 0.48 mmol) and stirred under N₂ protection for 45 min, then followed by addition of dry ZnCl₂ (109 mg, 0.80 mmol). The mixture was warmed to RT and stirred for 30 min, followed by addition of 5-bromo-3-iodo-1-methylpyridin-2(1H)-one and chloro(2-dicyclohexylphosphino-2′,6′-dimethoxy-1,1′-biphenyl)[2-(2′-amino-1,1′-biphenyl)]palladium(II) (11 mg, 0.02 mmol). The resulting mixture was heated to 50° C. and stirred for 2 h. The mixture was cooled, diluted with ethyl acetate, washed with saturated NH₄Cl and brine, dried (Na₂SO₄), filtered and the solvent was evaporated under reduced pressure. The resulting residue was purified by column chromatography (eluted with 0-4% MeOH/DCM) to provide 5-bromo-3-(furo[3,2-b]pyridin-2-yl)-1-methylpyridin-2(1H)-one (20 mg, yield: 21%). MS (M+H)⁺: 306.

### Step 2—Synthesis of 2-(4-fluorophenyl)-5-(5-(furo[3,2-b]pyridin-2-yl)-1-methyl-6-oxo-1,6-dihydropyridin-3-yl)-N-methyl-6-(N-methylmethylsulfonamido)benzofuran-3-carboxamide

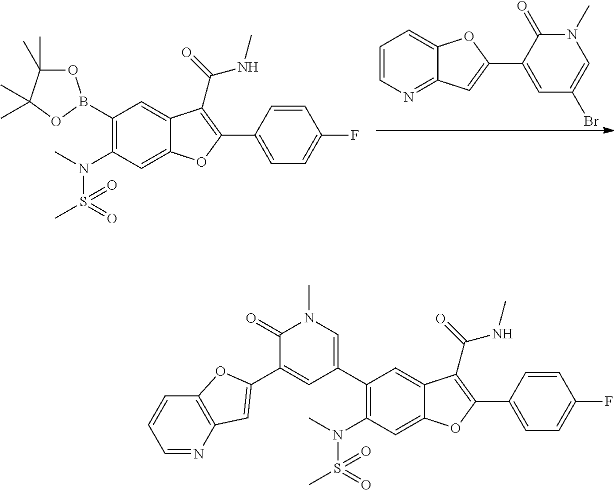 [SMILES: CNC(=O)c1c(-c2ccc(F)cc2)oc2cc(N(C)S(C)(=O)=O)c(B3OC(C)(C)C(C)(C)O3)cc12 ; Cn1cc(Br)cc(-c2cc3ncccc3o2)c1=O ; CNC(=O)c1c(-c2ccc(F)cc2)oc2cc(N(C)S(C)(=O)=O)c(-c3cc(-c4cc5ncccc5o4)c(=O)n(C)c3)cc12]

To a degassed solution of 2-(4-fluorophenyl)-N-methyl-6-(N-methylmethylsulfonamido)-5-(4,4,5,5-tetramethyl-1,3,2-dioxaborolan-2-yl)benzofuran-3-carboxamide (49 mg, 0.10 mmol) and 5-bromo-3-(furo[3,2-b]pyridin-2-yl)-1-methylpyridin-2(1H)-one (20 mg, 0.07 mmol) in 1,4-dioxane (1.5 mL) and water (100 μl) was added CS₂CO₃ (43 mg, 0.13 mmol) and 1,1′-bis(di-tert-butylphosphino)ferrocene palladium dichloride (8.5 mg, 0.01 mmol) under N₂ protection. The resulting mixture was heated to 90° C. and stirred at this temperature for 4 h. The reaction was cooled, filtered through a pad of the celite and washed with ethyl acetate. The combined filtrate was evaporated in vacuo. The resulting residue was purified by preparative TLC (eluted with 5% MeOH/DCM) to provide 2-(4-fluorophenyl)-5-(5-(furo[3,2-b]pyridin-2-yl)-1-methyl-6-oxo-1,6-dihydropyridin-3-yl)-N-methyl-6-(N-methylmethylsulfonamido)benzofuran-3-carboxamide (18 mg, yield: 46%). MS (M+H)⁺: 601.

Example 10, depicted in the table below, was prepared using the method described above.

| | | | MS |
|---|---|---|---|
| Example | Structure | IUPAC Name | (M + H)⁺ |
| 10 | 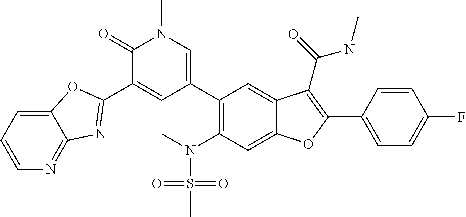 [SMILES: CNC(=O)c1c(-c2ccc(F)cc2)oc2cc(N(C)S(C)(=O)=O)c(-c3cc(-c4nc5ncccc5o4)c(=O)n(C)c3)cc12] | 2-(4-fluorophenyl)-N-methyl- 5-(1-methyl-5-(oxazolo[4,5- b]pyridin-2-yl)-6-oxo-1,6- dihydropyridin-3-yl)-6-(N- methylmethylsulfonamido) benzofuran-3-carboxamide | 602 |

### Example 11

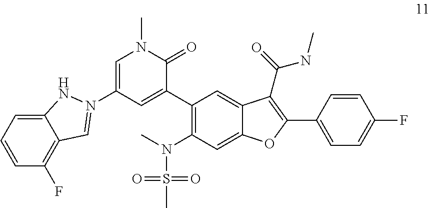

### Step 1—Synthesis of 5-(5-bromo-1-methyl-2-oxo-1,2-dihydropyridin-3-yl)-2-(4-fluorophenyl)-N-methyl-6-(N-methylmethylsulfonamido)benzofuran-3-carboxamide

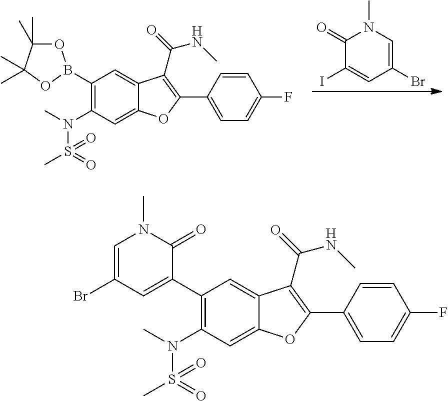

To a degassed solution of 2-(4-fluorophenyl)-N-methyl-6-(N-methylmethylsulfonamido)-5-(4,4,5,5-tetramethyl-1,3,2-dioxaborolan-2-yl)benzofuran-3-carboxamide (150 mg, 0.30 mmol) and 5-bromo-3-iodo-1-methylpyridin-2(1H)-one (100 mg, 0.32 mmol) in 1,4-dioxane (4 mL) and water (200 μl) was added K₂CO₃ (83 mg, 0.60 mmol) and pd(dppf)Cl₂ (22 mg, 0.03 mmol) under N₂ protection. The resulting mixture was heated to 50° C. and stirred at this temperature for 4 h. The reaction was cooled, filtered through a pad of the celite and washed with ethyl acetate. The combined filtrate was evaporated in vacuo. The resulting residue was purified using column chromatography (eluted with 0-100% EtOAc/hexane) to provide 5-(5-bromo-1-methyl-2-oxo-1,2-dihydropyridin-3-yl)-2-(4-fluorophenyl)-N-methyl-6-(N-methylmethylsulfonamido)benzofuran-3-carboxamide (120 mg, yield: 71%). MS (M+H)⁺: 563.

### Step 2—Synthesis of 5-(5-(4-fluoro-1H-indol-2-yl)-1-methyl-2-oxo-1,2-dihydropyridin-3-yl)-2-(4-fluorophenyl)-N-methyl-6-(N-methylmethylsulfonamido)benzofuran-3-carboxamide

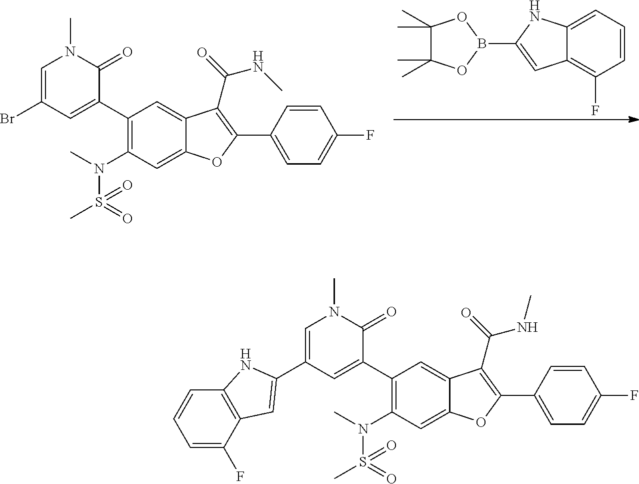 [SMILES: CNC(=O)c1c(-c2ccc(F)cc2)oc2cc(N(C)S(C)(=O)=O)c(-c3cc(Br)cn(C)c3=O)cc12 ; CC1(C)OB(c2cc3c(F)cccc3[nH]2)OC1(C)C ; CNC(=O)c1c(-c2ccc(F)cc2)oc2cc(N(C)S(C)(=O)=O)c(-c3cc(-c4cc5c(F)cccc5[nH]4)cn(C)c3=O)cc12]

To a solution of 5-(5-bromo-1-methyl-2-oxo-1,2-dihydropyridin-3-yl)-2-(4-fluorophenyl)-N-methyl-6-(N-methylmethylsulfonamido)benzofuran-3-carboxamide (50 mg, 0.09 mmol) and 4-fluoro-2-(4,4,5,5-tetramethyl-1,3,2-dioxaborolan-2-yl)-1H-indole (35 mg, 0.13 mmol) in 1,4-dioxane (2 mL) and water (100 μl) was added CS₂CO₃ (56 mg, 0.18 mmol) and 1,1′-bis(di-tert-butylphosphino)ferrocene palladium dichloride (12 mg, 0.02 mmol) under N₂ protection. The resulting mixture was heated to 80° C. and stirred at this temperature for 3 h. The reaction was cooled, filtered through a pad of the celite and washed with ethyl acetate. The combined filtrate was evaporated in vacuo. The resulting residue was purified by preparative TLC (eluted with 4% MeOH/DCM) to provide 5-(5-(4-fluoro-1H-indol-2-yl)-1-methyl-2-oxo-1,2-dihydropyridin-3-yl)-2-(4-fluorophenyl)-N-methyl-6-(N-methylmethylsulfonamido)benzofuran-3-carboxamide (14 mg, yield: 25%). MS (M+H)⁺: 617.

Example 12, depicted in the table below, was prepared using the method described above.

| | | | MS |
|---|---|---|---|
| Example | Structure | IUPAC Name | (M + H)⁺ |
| 12 | 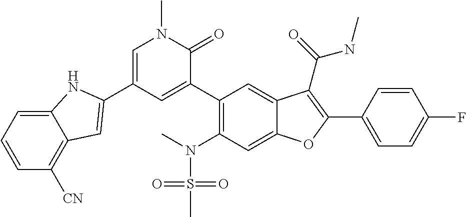 [SMILES: CNC(=O)c1c(-c2ccc(F)cc2)oc2cc(N(C)S(C)(=O)=O)c(-c3cc(-c4cc5c(C#N)cccc5[nH]4)cn(C)c3=O)cc12] | 5-(5-(4-cyano-1H-indol-2-yl)- 1-methyl-2-oxo-1,2- dihydropyridin-3-yl)-2-(4- fluorophenyl)-N-methyl-6-(N- methylmethylsulfonamido) benzofuran-3-carboxamide | 624 |

### Example 13

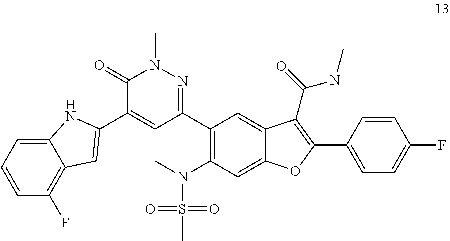 [SMILES: CNC(=O)c1c(-c2ccc(F)cc2)oc2cc(N(C)S(C)(=O)=O)c(-c3cc(-c4cc5c(F)cccc5[nH]4)c(=O)n(C)n3)cc12]

### Step 1—Synthesis of 5-(5-chloro-1-methyl-6-oxo-1,6-dihydropyridazin-3-yl)-2-(4-fluorophenyl)-N-methyl-6-(N-methylmethylsulfonamido)benzofuran-3-carboxamide

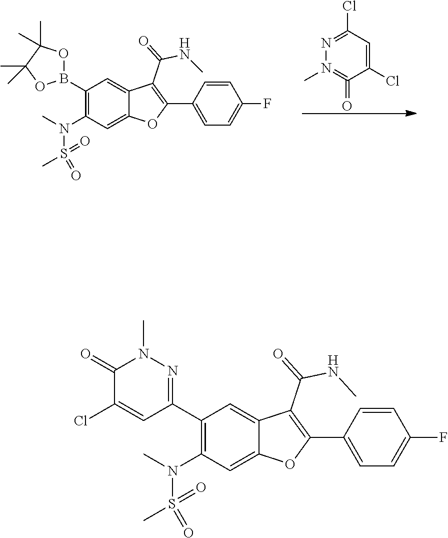

To a degassed solution of 2-(4-fluorophenyl)-N-methyl-6-(N-methylmethylsulfonamido)-5-(4,4,5,5-tetramethyl-1,3,2-dioxaborolan-2-yl)benzofuran-3-carboxamide (400 mg, 0.80 mmol) and 4,6-dichloro-2-methylpyridazin-3(2H)-one (214 mg, 1.19 mmol) in 1,4-dioxane (8 mL) and water (300 μl) was added K₂CO₃ (220 mg, 1.19 mmol) and pd(dppf)Cl₂ (117 mg, 0.06 mmol) under N₂ protection. The resulting mixture was heated to 70° C. and stirred at this temperature overnight. The reaction was cooled, filtered through a pad of the celite and washed with ethyl acetate. The combined filtrate was evaporated in vacuo. The resulting residue was purified by column chromatography (eluted with 0-100% EtOAc/hexane) to provide 5-(5-chloro-1-methyl-6-oxo-1,6-dihydropyridazin-3-yl)-2-(4-fluorophenyl)-N-methyl-6-(N-methylmethylsulfonamido)benzofuran-3-carboxamide (250 mg, yield: 60%). MS (M+H)⁺: 519.

### Step 2—Synthesis of 5-(5-(4-fluoro-1H-indol-2-yl)-1-methyl-6-oxo-1,6-dihydropyridazin-3-yl)-2-(4-fluorophenyl)-N-methyl-6-(N-methylmethylsulfonamido)benzofuran-3-carboxamide

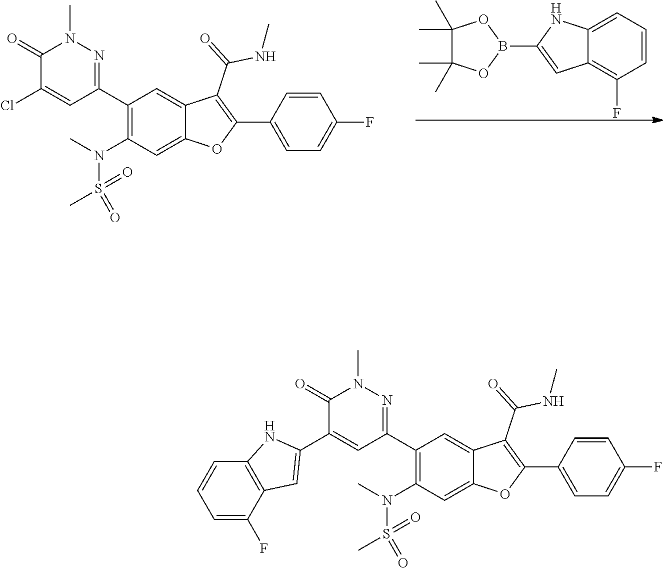 [SMILES: CNC(=O)c1c(-c2ccc(F)cc2)oc2cc(N(C)S(C)(=O)=O)c(-c3cc(Cl)c(=O)n(C)n3)cc12 ; CC1(C)OB(c2cc3c(F)cccc3[nH]2)OC1(C)C ; CNC(=O)c1c(-c2ccc(F)cc2)oc2cc(N(C)S(C)(=O)=O)c(-c3cc(-c4cc5c(F)cccc5[nH]4)c(=O)n(C)n3)cc12]

To a solution of 5-(5-chloro-1-methyl-6-oxo-1,6-dihydropyridazin-3-yl)-2-(4-fluorophenyl)-N-methyl-6-(N-methylmethylsulfonamido)benzofuran-3-carboxamide (100 mg, 0.19 mmol) and 4-fluoro-2-(4,4,5,5-tetramethyl-1,3,2-dioxaborolan-2-yl)-1H-indole (77 mg, 0.29 mmol) in 1,4-dioxane (2.0 mL) and water (100 μl) was added CS₂CO₃ (126 mg, 0.39 mmol) and 1,1′-bis(di-tert-butylphosphino)ferrocene palladium dichloride (13 mg, 0.02 mmol) under N₂ protection. The resulting mixture was heated to 90° C. and stirred at this temperature overnight. The reaction was cooled, filtered through a pad of the celite and washed with ethyl acetate. The combined filtrate was evaporated in vacuo. The resulting residue was purified by preparative TLC (eluted with 4% MeOH/DCM) to provide 5-(5-(4-fluoro-1H-indol-2-yl)-1-methyl-6-oxo-1,6-dihydropyridin-3-yl)-2-(4-fluorophenyl)-N-methyl-6-(N-methylmethylsulfonamido)benzofuran-3-carboxamide (105 mg, yield: 87%). MS (M+H)⁺: 625.

Examples 14 and 15, depicted in the table below, were prepared using the method described above.

| | | | MS |
|---|---|---|---|
| Example | Structure | IUPAC Name | (M + H)⁺ |
| 14 | 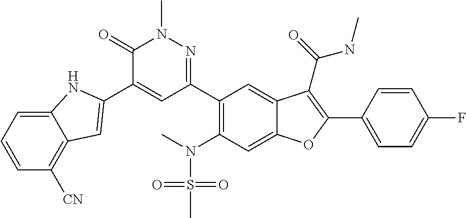 [SMILES: CNC(=O)c1c(-c2ccc(F)cc2)oc2cc(N(C)S(C)(=O)=O)c(-c3cc(-c4cc5c(C#N)cccc5[nH]4)c(=O)n(C)n3)cc12] | 5-(5-(4-cyano-1H-indol-2-yl)- 1-methyl-6-oxo-1,6- dihydropyridazin-3-yl)-2-(4- fluorophenyl)-N-methyl-6-(N- methylmethylsulfonamido) benzofuran-3-carboxamide | 625 |
| 15 | 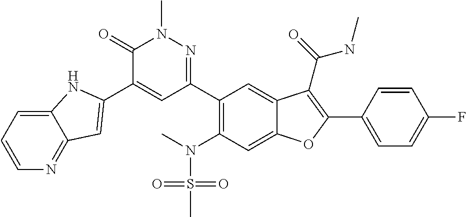 [SMILES: CNC(=O)c1c(-c2ccc(F)cc2)oc2cc(N(C)S(C)(=O)=O)c(-c3cc(-c4cc5ncccc5[nH]4)c(=O)n(C)n3)cc12] | 2-(4-fluorophenyl)-N-methyl- 5-(1-methyl-6-oxo-5-(1H- pyrrolo[3,2-b]pyridin-2-yl)- 1,6-dihydropyridazin-3-yl)-6- (N-methylmethylsulfonamido) benzofuran-3-carboxamide | 601 |

### Example 16

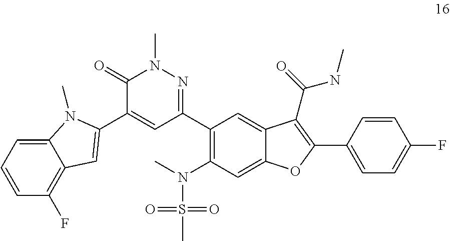 [SMILES: CNC(=O)c1c(-c2ccc(F)cc2)oc2cc(N(C)S(C)(=O)=O)c(-c3cc(-c4cc5c(F)cccc5n4C)c(=O)n(C)n3)cc12]

A mixture of 5-(5-(4-fluoro-1H-indol-2-yl)-1-methyl-6-oxo-1,6-dihydropyridazin-3-yl)-2-(4-fluorophenyl)-N-methyl-6-(N-methylmethylsulfonamido)benzofuran-3-carboxamide (45 mg, 0.07 mmol), MeI (52 mg, 0.36 mmol) and Cs₂CO₃ (95 mg, 0.29 mmol) in DMF (4 ml) was stirred at RT for 2 h. Filtration through a pad of celite removed the solid. After being washed with ethyl acetate, the combined filtrate was concentrated in vacuo. The resulting residue was purified by preparative TLC (eluted with ethyl acetate) to provide 5-(5-(4-fluoro-1-methyl-1H-indol-2-yl)-1-methyl-6-oxo-1,6-dihydropyridazin-3-yl)-2-(4-fluorophenyl)-N-methyl-6-(N-methylmethylsulfonamido)benzofuran-3-carboxamide (32 mg, yield: 71%). MS (M+H)⁺: 632.

Example 17, depicted in the table below, was prepared using the method described above.

| | | | MS |
|---|---|---|---|
| Example | Structure | IUPAC Name | (M + H)⁺ |
| 17 | 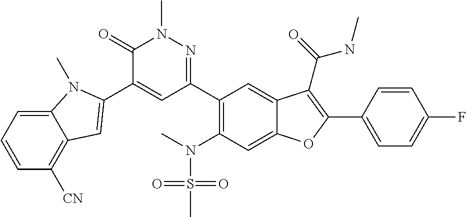 [SMILES: CNC(=O)c1c(-c2ccc(F)cc2)oc2cc(N(C)S(C)(=O)=O)c(-c3cc(-c4cc5c(C#N)cccc5n4C)c(=O)n(C)n3)cc12] | 5-(5-(4-cyano-1-methyl-1H- indol-2-yl)-1-methyl-6-oxo- 1,6-dihydropyridazin-3-yl)-2- (4-fluorophenyl)-N-methyl-6- (N- methylmethylsulfonamido) benzofuran-3-carboxamide | 639 |

### Example 18

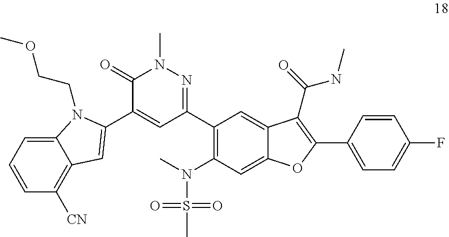 [SMILES: CNC(=O)c1c(-c2ccc(F)cc2)oc2cc(N(C)S(C)(=O)=O)c(-c3cc(-c4cc5c(C#N)cccc5n4CCOC)c(=O)n(C)n3)cc12]

A mixture of 5-(5-(4-cyano-1H-indol-2-yl)-1-methyl-6-oxo-1,6-dihydropyridazin-3-yl)-2-(4-fluorophenyl)-N-methyl-6-(N-methylmethylsulfonamido)benzofuran-3-carboxamide (50 mg, 0.08 mmol), 1-iodo-2-methoxyethane (29 mg, 0.16 mmol) and Cs₂CO₃ (102 mg, 0.31 mmol) in DMF (1.5 ml) was stirred at RT for 4 h. Filtration through a pad of celite removed the solid. After being washed with ethyl acetate, the combined filtrate was concentrated in vacuo. The resulting residue was purified by preparative TLC (eluted with 5% MeOH/DCM) to provide 5-(5-(4-cyano-1-(2-methoxyethyl)-1H-indol-2-yl)-1-methyl-6-oxo-1,6-dihydropyridazin-3-yl)-2-(4-fluorophenyl)-N-methyl-6-(N-methylmethylsulfonamido)benzofuran-3-carboxamide (17, 30 mg, yield: 56%). MS (M+H)⁺: 683.

### Example 19

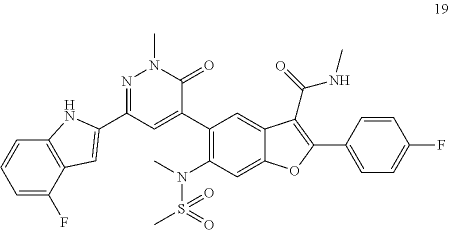 [SMILES: CNC(=O)c1c(-c2ccc(F)cc2)oc2cc(N(C)S(C)(=O)=O)c(-c3cc(-c4cc5c(F)cccc5[nH]4)nn(C)c3=O)cc12]

### Step 1—Synthesis of 4-chloro-6-(4-fluoro-1H-indol-2-yl)-2-methylpyridazin-3(2H)-one

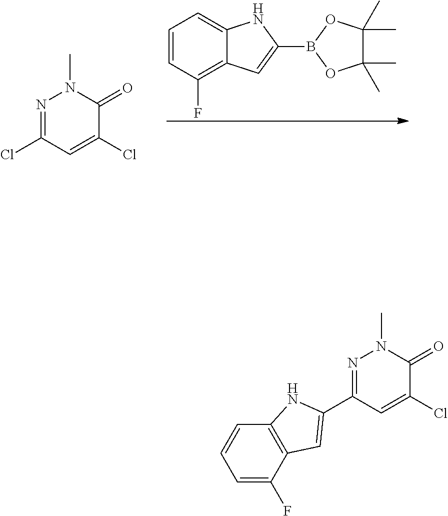

To a degassed solution of 4-fluoro-2-(4,4,5,5-tetramethyl-1,3,2-dioxaborolan-2-yl)-1H-indole (193 mg, 0.74 mmol) and 4,6-dichloro-2-methylpyridazin-3(2H)-one (120 mg, 0.67 mmol) in 1,4-dioxane (3.0 mL) and water (200 μl) was added Cs₂CO₃ (437 mg, 1.34 mmol) and 1,1′-bis(di-tert-butylphosphino)ferrocene palladium dichloride (44 mg, 0.07 mmol) under N₂ protection. The resulting mixture was heated to 80° C. and stirred at this temperature overnight. The reaction was cooled, filtered through a pad of celite and washed with ethyl acetate. The combined filtrate was evaporated in vacuo. The resulting residue was purified by preparative TLC (eluted with 0-3% MeOH/DCM) to provide 4-chloro-6-(4-fluoro-1H-indol-2-yl)-2-methylpyridazin-3(2H)-one (120 mg, yield: 64%). MS (M+H)⁺: 278.

### Step 2—Synthesis of 5-(6-(4-fluoro-1H-indol-2-yl)-2-methyl-3-oxo-2,3-dihydropyridazin-4-yl)-2-(4-fluorophenyl)-N-methyl-6-(N-methylmethylsulfonamido)benzofuran-3-carboxamide

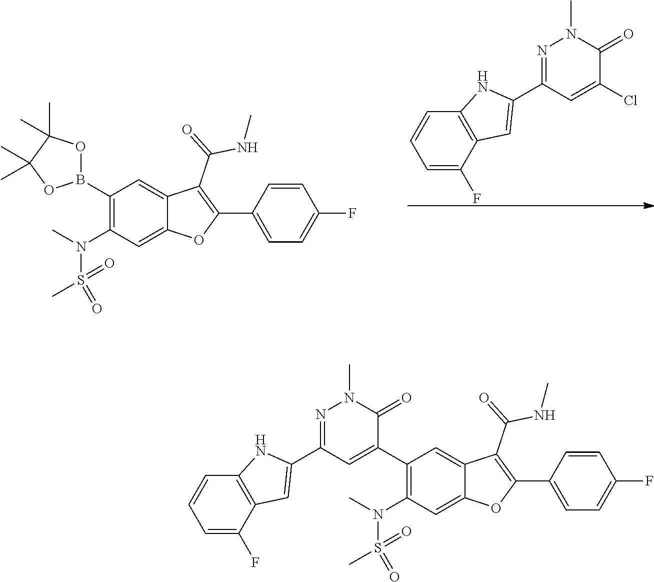 [SMILES: CNC(=O)c1c(-c2ccc(F)cc2)oc2cc(N(C)S(C)(=O)=O)c(B3OC(C)(C)C(C)(C)O3)cc12 ; Cn1nc(-c2cc3c(F)cccc3[nH]2)cc(Cl)c1=O ; CNC(=O)c1c(-c2ccc(F)cc2)oc2cc(N(C)S(C)(=O)=O)c(-c3cc(-c4cc5c(F)cccc5[nH]4)nn(C)c3=O)cc12]

To a solution of 2-(4-fluorophenyl)-N-methyl-6-(N-methylmethylsulfonamido)-5-(4,4,5,5-tetramethyl-1,3,2-dioxaborolan-2-yl)benzofuran-3-carboxamide (120 mg, 0.24 mmol) and 4-chloro-6-(4-fluoro-1H-indol-2-yl)-2-methylpyridazin-3(2H)-one (99 mg, 0.36 mmol) in 1,4-dioxane (4.0 mL) and water (200 μl) was added CS₂CO₃ (156 mg, 0.48 mmol) and 1,1′-bis(di-tert-butylphosphino)ferrocene palladium dichloride (16 mg, 0.024 mmol) under N₂ protection. The resulting mixture was heated to 90° C. and stirred at this temperature overnight. The reaction was cooled, filtered through a pad of the celite and washed with ethyl acetate. The combined filtrate was evaporated in vacuo. The resulting residue was purified using preparative TLC (eluted with 4% MeOH/DCM) to provide 5-(6-(4-fluoro-1H-indol-2-yl)-2-methyl-3-oxo-2,3-dihydropyridazin-4-yl)-2-(4-fluorophenyl)-N-methyl-6-(N-methylmethylsulfonamido)benzofuran-3-carboxamide (15 mg, yield: 10%). MS (M+H)⁺: 618.

### Example 20

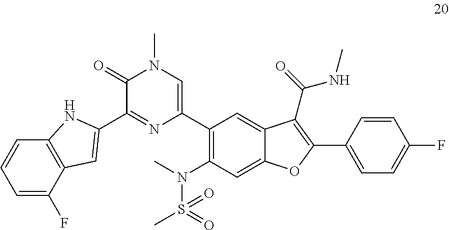 [SMILES: CNC(=O)c1c(-c2ccc(F)cc2)oc2cc(N(C)S(C)(=O)=O)c(-c3cn(C)c(=O)c(-c4cc5c(F)cccc5[nH]4)n3)cc12]

### Step 1—Synthesis of tert-butyl 2-(6-bromo-3-hydroxypyrazin-2-yl)-7-fluoro-1H-indole-1-carboxylate

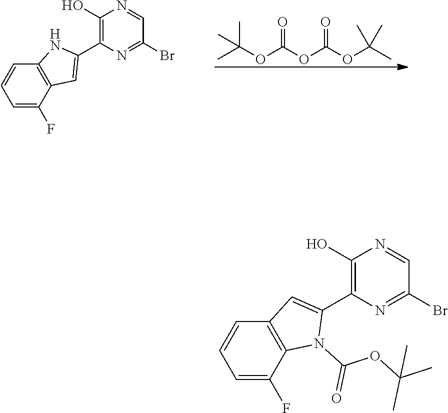 [SMILES: Oc1ncc(Br)nc1-c1cc2c(F)cccc2[nH]1 ; CC(C)(C)OC(=O)OC(=O)OC(C)(C)C ; CC(C)(C)OC(=O)n1c(-c2nc(Br)cnc2O)cc2cccc(F)c21]

A mixture of 5-bromo-3-(4-fluoro-1H-indol-2-yl)pyrazin-2-ol (400 mg, 1.30 mmol) and di-tert-butyl dicarbonate (368 mg, 1.69 mmol) in THF (10 ml) was stirred at RT overnight. The volatiles were removed in vacuo. The resulting residue was purified by column chromatography (eluted with 20-80% ethyl acetate/hexane) to provide tert-butyl 2-(6-bromo-3-hydroxypyrazin-2-yl)-7-fluoro-1H-indole-1-carboxylate (500 mg, yield: 49%). MS (M+H)⁺: 409.

### Step 2—Synthesis of tert-butyl 2-(6-bromo-4-methyl-3-oxo-3,4-dihydropyrazin-2-yl)-7-fluoro-1H-indole-1-carboxylate

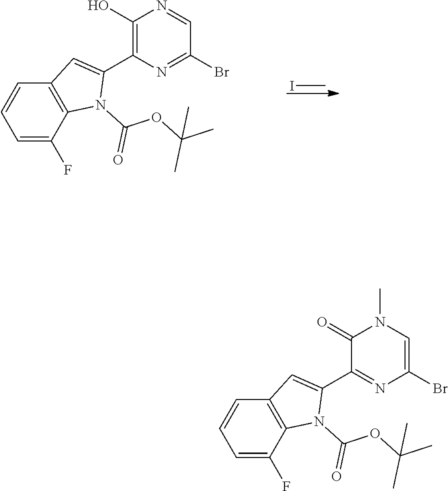

A mixture of tert-butyl 2-(6-bromo-3-hydroxypyrazin-2-yl)-7-fluoro-1H-indole-1-carboxylate (200 mg, 0.49 mmol), MeI (139 mg, 0.98 mmol) and Cs₂CO₃ (319 mg, 0.98 mmol) in DMF (3 ml) was stirred at RT for 4 h. Filtration through a pad of celite removed the solid. After being washed with ethyl acetate, the combined filtrate was concentrated in vacuo. The resulting residue was purified by preparative TLC (eluted with DCM) to provide tert-butyl 2-(6-bromo-4-methyl-3-oxo-3,4-dihydropyrazin-2-yl)-7-fluoro-1H-indole-1-carboxylate (150 mg, yield: 72%). MS (M+H)⁺: 423.

### Step 3—Synthesis of tert-butyl 4-fluoro-2-(6-(2-(4-fluorophenyl)-3-(methylcarbamoyl)-6-(N-methylmethylsulfonamido)benzofuran-5-yl)-4-methyl-3-oxo-3,4-dihydropyrazin-2-yl)-1H-indole-1-carboxylate

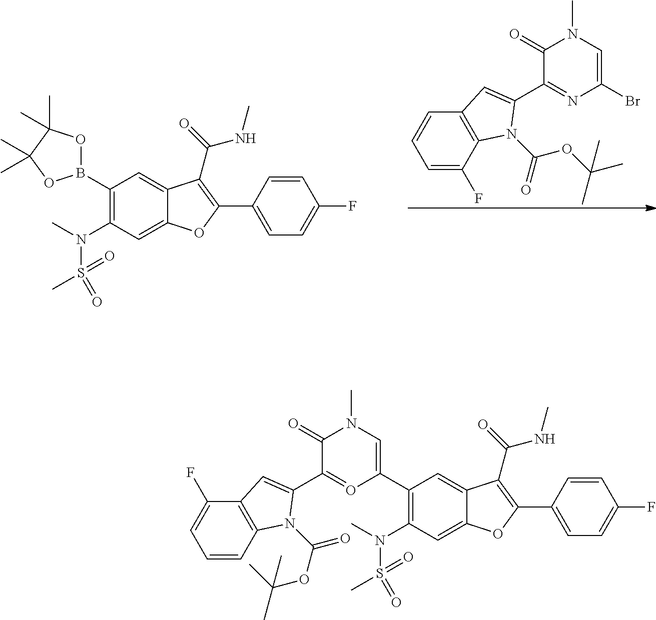

To a solution of 2-(4-fluorophenyl)-N-methyl-6-(N-methylmethylsulfonamido)-5-(4,4,5,5-tetramethyl-1,3,2-dioxaborolan-2-yl)benzofuran-3-carboxamide (214 mg, 0.43 mmol) and tert-butyl 2-(6-bromo-4-methyl-3-oxo-3,4-dihydropyrazin-2-yl)-7-fluoro-1H-indole-1-carboxylate (150 mg, 0.35 mmol) in 1,4-dioxane (3.0 mL) and water (200 μl) was added CS₂CO₃ (231 mg, 0.71 mmol) and 1,1′-bis(di-tert-butylphosphino)ferrocene palladium dichloride (23 mg, 0.036 mmol) under N₂ protection. The resulting mixture was heated to 80° C. and stirred at this temperature overnight. The reaction was cooled, filtered through a pad of celite and washed with ethyl acetate. The combined filtrate was evaporated in vacuo. The resulting residue was purified by column chromatography (eluted with 0-34% MeOH/DCM) to provide tert-butyl 4-fluoro-2-(6-(2-(4-fluorophenyl)-3-(methylcarbamoyl)-6-(N-methylmethylsulfonamido)benzofuran-5-yl)-4-methyl-3-oxo-3,4-dihydropyrazin-2-yl)-1H-indole-1-carboxylate (180 mg, yield: 71%). MS (M+H)⁺: 718.

### Step 4—Synthesis of 5-(6-(4-fluoro-1H-indol-2-yl)-4-methyl-5-oxo-4,5-dihydropyrazin-2-yl)-2-(4-fluorophenyl)-N-methyl-6-(N-methylmethylsulfonamido)benzofuran-3-carboxamide

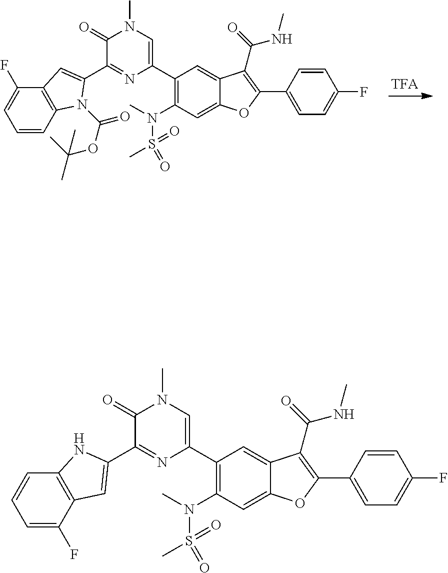 [SMILES: CNC(=O)c1c(-c2ccc(F)cc2)oc2cc(N(C)S(C)(=O)=O)c(-c3cn(C)c(=O)c(-c4cc5c(F)cccc5n4C(=O)OC(C)(C)C)n3)cc12 ; CNC(=O)c1c(-c2ccc(F)cc2)oc2cc(N(C)S(C)(=O)=O)c(-c3cn(C)c(=O)c(-c4cc5c(F)cccc5[nH]4)n3)cc12]

A mixture of tert-butyl 4-fluoro-2-(6-(2-(4-fluorophenyl)-3-(methylcarbamoyl)-6-(N-methylmethylsulfonamido)benzofuran-5-yl)-4-methyl-3-oxo-3,4-dihydropyrazin-2-yl)-1H-indole-1-carboxylate (150 mg, 0.21 mmol) and TFA (2.0 mL) was stirred at RT for 30 min and concentrated in vacuo. The residue was suspended in saturated NaHCO₃ and extracted with DCM. The organic phase was dried over Na₂SO₄ and concentrated in vacuum. The residue was purified by column chromatography (eluted with 0-3% MeOH/DCM) to give 5-(6-(4-fluoro-1H-indol-2-yl)-4-methyl-5-oxo-4,5-dihydropyrazin-2-yl)-2-(4-fluorophenyl)-N-methyl-6-(N-methylmethylsulfonamido)benzofuran-3-carboxamide (110 mg, yield: 85%). MS (M+H)⁺: 618.

### Example 21

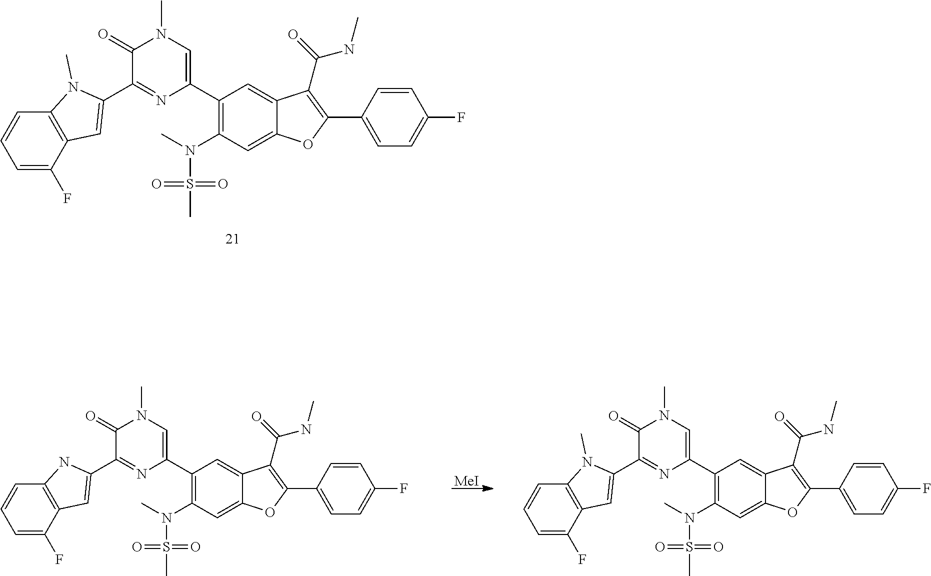

A mixture of 5-(6-(4-fluoro-1H-indol-2-yl)-4-methyl-5-oxo-4,5-dihydropyrazin-2-yl)-2-(4-fluorophenyl)-N-methyl-6-(N-methylmethylsulfonamido)benzofuran-3-carboxamide (30 mg, 0.05 mmol), MeI (28 mg, 0.19 mmol) and Cs₂CO₃ (63 mg, 0.19 mmol) in DMF (1 ml) was stirred at RT for 2 h. Filtration through a pad of celite removed the solid. After being washed with ethyl acetate, the combined filtrate was concentrated in vacuo. The resulting residue was purified by preparative TLC (eluted with ethyl acetate) to provide 5-(6-(4-fluoro-1-methyl-1H-indol-2-yl)-4-methyl-5-oxo-4,5-dihydropyrazin-2-yl)-2-(4-fluorophenyl)-N-methyl-6-(N-methylmethylsulfonamido)benzofuran-3-carboxamide (23 mg, yield: 75%). MS (M+H)⁺: 632.

### Example 22

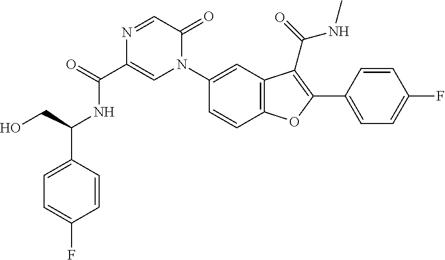 [SMILES: CNC(=O)c1c(-c2ccc(F)cc2)oc2ccc(-n3cc(C(=O)N[C@H](CO)c4ccc(F)cc4)ncc3=O)cc12]

### Step 1: Synthesis of methyl 4-(2-(4-fluorophenyl)-3-(methylcarbamoyl)benzofuran-5-yl)-5-oxo-4,5-dihydropyrazine-2-carboxylate

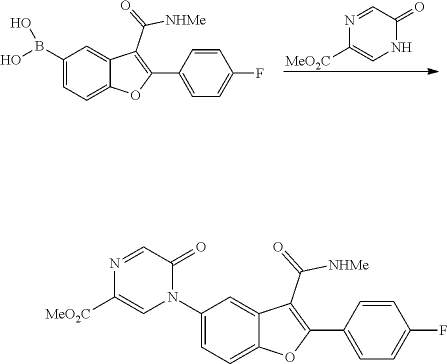 [SMILES: CNC(=O)c1c(-c2ccc(F)cc2)oc2ccc(B(O)O)cc12 ; COC(=O)c1c[nH]c(=O)cn1 ; CNC(=O)c1c(-c2ccc(F)cc2)oc2ccc(-n3cc(C(=O)OC)ncc3=O)cc12]

To a solution of (2-(4-fluorophenyl)-3-(methylcarbamoyl)benzofuran-5-yl)boronic acid (0.79 g, 2.52 mmol) in 25 ml MeOH was added Cu(OTf)₂ (0.913 g, 2.52 mmol), methyl 5-oxo-4,5-dihydropyrazine-2-carboxylate (0.389 g, 2.52 mmol), followed by pyridine (0.408 mL, 3.99 mmol). The mixture was stirred at ambient temp for 16 h. The mixture was concentrated. It was partitioned between 1N HCl 20 mL and 20 mL DCM. The organic layer was separated and the aqueous layer was extracted with 20 mL DCM 3 times. The combined organic layers were dried and concentrated. Product was obtained as white solid (770 mg, yield 72.4%) and used as is in next step. Exact Mass [M+H]+(422).

### Step 2: Synthesis of 4-(2-(4-fluorophenyl)-3-(methylcarbamoyl)benzofuran-S-yl)-5-oxo-4,5-dihydropyrazine-2-carboxylic acid

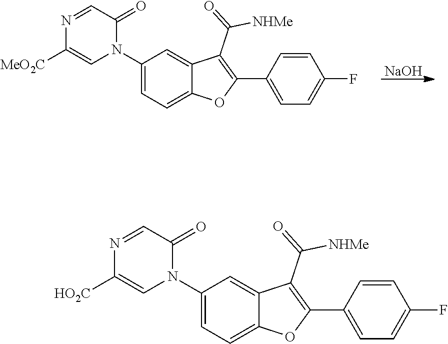

To a solution of methyl 4-(2-(4-fluorophenyl)-3-(methylcarbamoyl)benzofuran-5-yl)-5-oxo-4,5-dihydropyrazine-2-carboxylate ester (750 mg, 1.78 mmol) in 4 mL MeOH and 8 mL of THF was added 1N NaOH 5.34 mL. The mixture was stirred for 30 min. LCMS showed completion of the reaction. The reaction was neutralized with addition of 1N HCl, and then the volatiles were removed by rotavap. The aqueous layer was extracted with DCM 15 mL 3 times. The combined organic layer was dried over Na₂SO₄, filtered and concentrated. The resulting product (350 mg, yield 48.3%) was used in the next step without further purification. Exact Mass [M+H]+ (408).

### Step 3: Synthesis of (S)-N-(1-(4-fluorophenyl)-2-hydroxyethyl)-4-(2-(4-fluorophenyl)-3-(methylcarbamoyl)benzofuran-5-yl)-5-oxo-4,5-dihydropyrazine-2-carboxamide4-(2-(4-fluorophenyl)-3-(methylcarbamoyl)benzofuran-5-yl)-5-oxo-4,5-dihydropyrazine-2-carboxylic acid

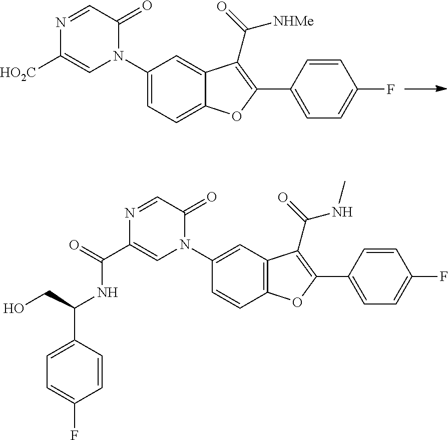 [SMILES: CNC(=O)c1c(-c2ccc(F)cc2)oc2ccc(-n3cc(C(=O)O)ncc3=O)cc12 ; CNC(=O)c1c(-c2ccc(F)cc2)oc2ccc(-n3cc(C(=O)N[C@H](CO)c4ccc(F)cc4)ncc3=O)cc12]

To a solution of 4-(2-(4-fluorophenyl)-3-(methylcarbamoyl)benzofuran-5-yl)-5-oxo-4,5-dihydropyrazine-2-carboxylic acid (40 mg) in 1 mL DMF was added sequentially (S)-2-amino-2-(4-fluorophenyl)ethanol (15.4 mg, 0.098 mmol), DIEA (0.069 mL) followed by 0.25 mL T3P (50% wt in EtOAc). The mixture was stirred for 16 h overnight. After normal work up, the residue was purified using semi-prep HPLC to give the desired product. Exact mass [M+H]+(545).

Examples 23-26, depicted in the table below, were prepared using the method described above.

| | | | MS |
|---|---|---|---|
| Example | Structure | IUPAC Name | (M + H)⁺ |
| 23 | 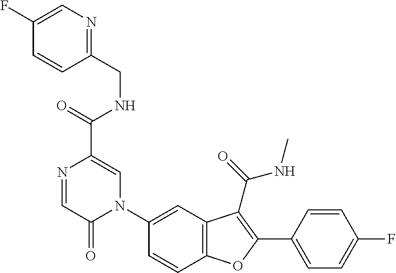 [SMILES: CNC(=O)c1c(-c2ccc(F)cc2)oc2ccc(-n3cc(C(=O)NCc4ccc(F)cn4)ncc3=O)cc12] | 4-(2-(4-fluorophenyl)-3- (methylcarbamoyl) benzofuran- 5-yl)-N-((5-fluoropyridin- 2-yl)methyl)-5-oxo-4,5- dihydropyrazine-2- carboxamide | 516 |
| 24 | 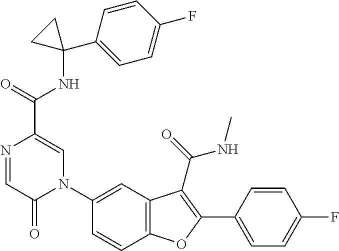 [SMILES: CNC(=O)c1c(-c2ccc(F)cc2)oc2ccc(-n3cc(C(=O)NC4(c5ccc(F)cc5)CC4)ncc3=O)cc12] | 4-(2-(4-fluorophenyl)-3- (methylcarbamoyl) benzofuran- 5-yl)-N-(1-(4- fluorophenyl)cyclopropyl)- 5-oxo-4,5-dihydropyrdzine- 2-carboxamide | 541 |
| 25 | 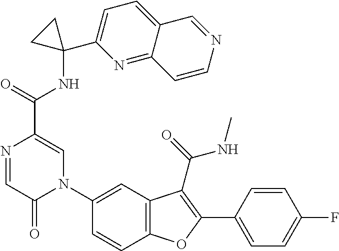 [SMILES: CNC(=O)c1c(-c2ccc(F)cc2)oc2ccc(-n3cc(C(=O)NC4(c5ccc6cnccc6n5)CC4)ncc3=O)cc12] | N-(1-(1,6-naphthyridin-2- yl)cyclopropyl)-4-(2-(4- fluorophenyl)-3- (methylcarbamoyl) benzofuran- 5-yl)-5-oxo-4,5- dihydropyrazine-2- carboxamide | 575 |
| 26 | 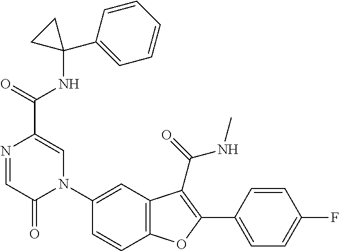 [SMILES: CNC(=O)c1c(-c2ccc(F)cc2)oc2ccc(-n3cc(C(=O)NC4(c5ccccc5)CC4)ncc3=O)cc12] | 4-(2-(4-fluorophenyl)-3- (methylcarbamoyl) benzofuran- 5-yl)-5-oxo-N-(1- phenylcyclopropyl)-4,5- dihydropyrazine-2- carboxamide | 523 |
| 27 | 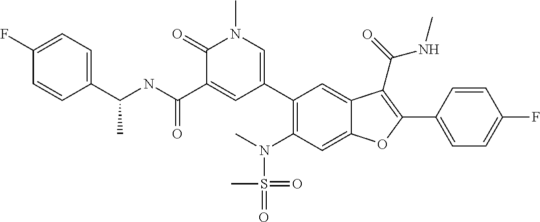 [SMILES: CNC(=O)c1c(-c2ccc(F)cc2)oc2cc(N(C)S(C)(=O)=O)c(-c3cc(C(=O)N[C@H](C)c4ccc(F)cc4)c(=O)n(C)c3)cc12] | (R)-5-(2-(4-fluorophenyl)-3- (methylcarbamoyl)-6-(N- methylmethylsulfonamido) benzoturan-5-yl)-N-(1-(4- fluorophenyl)ethyl)- 1-methyl-2-oxo- 1,2-dihydropyridine-3- carboxamide | 649 |

### Example 28

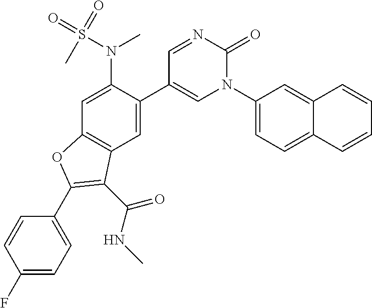 [SMILES: CNC(=O)c1c(-c2ccc(F)cc2)oc2cc(N(C)S(C)(=O)=O)c(-c3cnc(=O)n(-c4ccc5ccccc5c4)c3)cc12]

### Step 1: Synthesis of 5-bromo-1-(naphthalen-2-yl)pyrimidin-2(1H)-one

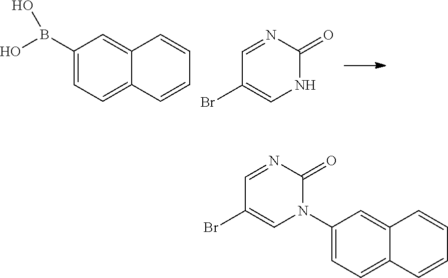

To 14 mL MeOH was added 5-bromopyrimidin-2(1H)-one (0.50 g, 2.86 mmol), naphthalen-2-ylboronic acid (0.59 g, 3.43 mmol) then Cu(OTf)₂ (1.03 g, 2.86 mmol), followed by pyridine 0.46 mL. The mixture was stirred at ambient temp for 16 h. The mixture was concentrated. The residue was partitioned between water (20 mL) and EtOAc (10 mL). The layers were separated and the organic layer was extracted with EtOAc 10 mL×2. The combined organic layer was concentrated and the residue was purified on silica 0-100% EtOA/Hexanes. The desired product was obtained (320 mg, yield 37.2%). Exact Mass [M+H]+ (302).

### Step 2: Synthesis of 2-(4-fluorophenyl)-N-methyl-6-(N-methylmethylsulfonamido)-5-(1-(naphthalen-2-yl)-2-oxo-1,2-dihydropyrimidin-5-yl)benzofuran-3-carboxamide

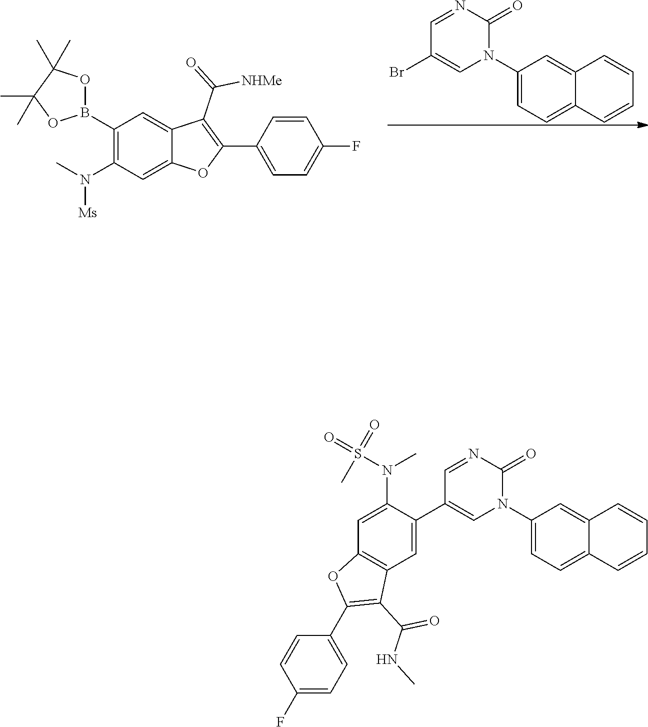 [SMILES: CNC(=O)c1c(-c2ccc(F)cc2)oc2cc(N(C)S(C)(=O)=O)c(B3OC(C)(C)C(C)(C)O3)cc12 ; O=c1ncc(Br)cn1-c1ccc2ccccc2c1 ; CNC(=O)c1c(-c2ccc(F)cc2)oc2cc(N(C)S(C)(=O)=O)c(-c3cnc(=O)n(-c4ccc5ccccc5c4)c3)cc12]

To a solution of 2-(4-fluorophenyl)-N-methyl-6-(N-methylmethylsulfonamido)-5-(4,4,5,5-tetramethyl-1,3,2-dioxaborolan-2-yl)benzofuran-3-carboxamide (30 mg, 0.06 mmol) in dioxane (1 mL) was added 5-bromo-1-(naphthalen-2-yl)pyrimidin-2(1H)-one (0.07 mmol, 1.2 eq.), Pd catalysts (2 mg, 0.05 eq.), K₃PO₄ (38 mg, 3 eq.) and H₂O (250 μl). The mixture was microwaved @ 100° C. for 4 h. The mixture was diluted with 3 mL EtOAc and 1 mL water. The mixtures were shaken for 10 min and the aq layer discarded. The organic layers were filtered through silicycle DMI cartridges then concentrated. The residue was purified using semi-prep HPLC. Exact mass [M+H]+(597).

Example 29, depicted in the table below, was prepared using the method described above.

| Compound | | | MS |
|---|---|---|---|
| ID | Structure | IUPAC Name | (M + H)⁺ |
| 29 | 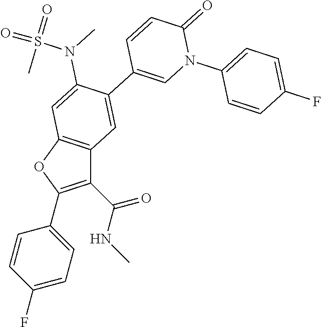 [SMILES: CNC(=O)c1c(-c2ccc(F)cc2)oc2cc(N(C)S(C)(=O)=O)c(-c3ccc(=O)n(-c4ccc(F)cc4)c3)cc12] | 2-(4-fluorophenyl)-5-(1-(4- fluorophenyl)-6-oxo-1,6- dihydropyridin-3-yl)-N- methyl-6-(N- methylmethylsulfonamido) benzofuran-3-carboxamide | 564 |

### Example 30

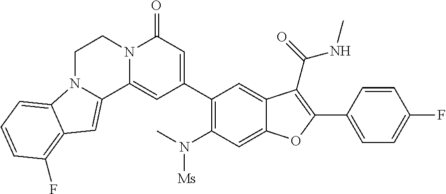 [SMILES: CNC(=O)c1c(-c2ccc(F)cc2)oc2cc(N(C)S(C)(=O)=O)c(-c3cc4n(c(=O)c3)CCn3c-4cc4c(F)cccc43)cc12]

### Step 1—Synthesis of tert-butyl 2-(6-amino-4-chloropyridin-2-yl)-4-fluoro-1H-indole-1-carboxylate

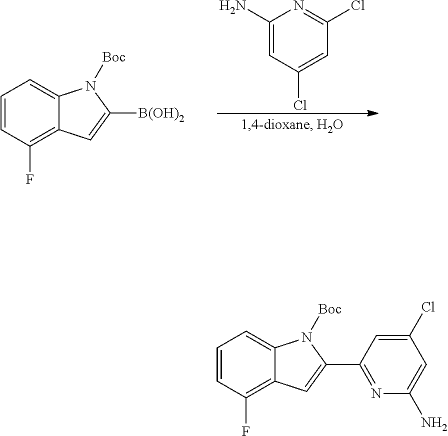 [SMILES: CC(C)(C)OC(=O)n1c(B(O)O)cc2c(F)cccc21 ; Nc1cc(Cl)cc(Cl)n1 ; CC(C)(C)OC(=O)n1c(-c2cc(Cl)cc(N)n2)cc2c(F)cccc21]

To a mixture of (1-(tert-butoxycarbonyl)-4-fluoro-1H-indol-2-yl)boronic acid (400 mg, 1.65 mmol), 4,6-dichloropyridin-2-amine (192 mg, 1.20 mmol) and Cs₂CO₃ (938 mg, 3.30 mmol) in 1,4-dioxane/water (6 mL/3 mL) was added Pd(PPh₃)₂Cl₂ (180 mg, 0.22 mmol) under nitrogen. The mixture was heated at 70° C. for 3 hours and concentrated in vacuum. After being extracted with EtOAc, the combined organic phase was washed with brine, dried over Na₂SO₄ and concentrated in vacuum. The residue was purified by column chromatography (PE:EA=10:1) to give tert-butyl 2-(6-amino-4-chloropyridin-2-yl)-4-fluoro-1H-indole-1-carboxylate (300 mg, yield: 70%). ¹H-NMR (CDCl₃, 400 MHz) δ 7.94 (d, J=8.4 Hz, 1H), 7.26 (s, 1H), 6.90˜6.95 (m, 2H), 6.83 (s, 1H), 6.52 (s, 1H), 1.39 (s, 9H). MS (M+H)⁺: 362.

### Step 2—Synthesis of 4-chloro-6-(4-fluoro-1H-indol-2-yl)pyridin-2-amine

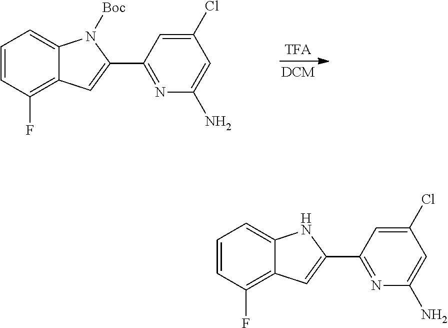 [SMILES: CC(C)(C)OC(=O)n1c(-c2cc(Cl)cc(N)n2)cc2c(F)cccc21 ; Nc1cc(Cl)cc(-c2cc3c(F)cccc3[nH]2)n1]

To a solution of tert-butyl 2-(6-amino-4-chloropyridin-2-yl)-4-fluoro-1H-indole-1-carboxylate (500 mg, 1.40 mmol) in DCM (10 mL) was added TFA (5 mL) under nitrogen at 0° C. The mixture was stirred for 12 hours, and then the mixture was adjusted to pH=7 with K₂CO₃. After being extracted with EA and concentrated, the residue was purified by prep-TLC (PE:EA=1:1) to give 4-chloro-6-(4-fluoro-1H-indol-2-yl)pyridin-2-amine (300 mg, yield: 83%). ¹H-NMR (CDCl₃, 400 MHz) δ 10.22 (s, 1H), 6.92˜7.02 (m, 2H), 6.89˜6.91 (m, 1H), 6.83 (s, 1H), 6.25 (s, 1H), 4.96 (s, 2H). MS (M+H)⁺: 262.

### Step 3—Synthesis of 4-chloro-6-(4-fluoro-1H-indol-2-yl)pyridin-2(1H)-one

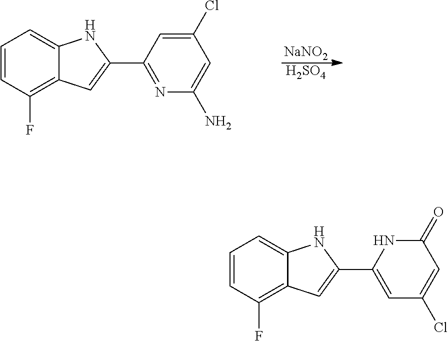

To a solution of 4-chloro-6-(4-fluoro-1H-indol-2-yl)pyridin-2-amine (300 mg, 1.15 mmol) in 5% H₂SO₄ (a.q., 15 mL) under nitrogen at 0° C. was added dropwise NaNO₂ (238 mg, 3.45 mmol, in 2 mL of H₂O). Then the mixture was stirred at 0° C. for 12 hours, filtered and extracted with EA. The combined organic phase was washed with brine, dried over Na₂SO₄ and concentrated. The residue was purified by chromatography (DCM:MeOH=20:1) to give 4-chloro-6-(4-fluoro-1H-indol-2-yl)pyridin-2(1H)-one (128 mg, yield: 41%). ¹H-NMR (DMSO-d₆, 400 MHz) δ 7.18˜7.24 (m, 2H), 7.04˜7.09 (m, 1H), 6.91 (s, 1H), 6.72˜6.76 (m, 1H), 6.16 (s, 1H). MS (M+H)⁺: 263.

### Step 4—Synthesis of 2-chloro-12-fluoro-6,7-dihydro-4H-pyrido[2′,1′:3,4]pyrazino[1,2-a]indol-4-one

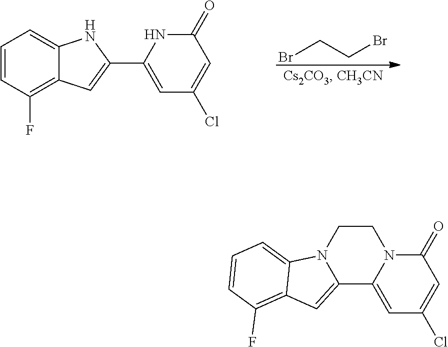

To a solution of 4-chloro-6-(4-fluoro-1H-indol-2-yl)pyridin-2(1H)-one (100 mg, 0.38 mmol), Cs₂CO₃ (373 mg, 1.14 mmol) in acetonitrile (10 mL) was added dropwise 1,2-dibromoethane (189 mg, 0.38 mmol) under nitrogen at 90° C. The mixture was stirred at 90° C. for 8 hours. After the solvent was removed in vacuum, the residue was extracted with EtOAc. The combined organic phase was washed with brine, dried over Na₂SO₄ and concentrated in vacuum. The residue was purified by prep-TLC (DCM:EtOAc=1:1) to give 2-chloro-12-fluoro-6,7-dihydro-4H-pyrido[2′,1′:3,4]pyrazino[1,2-a]indol-4-one (50 mg, yield: 45%). ¹H-NMR (CDCl₃, 400 MHz) δ 7.23 (s, 1H), 7.12˜7.16 (m, 2H), 6.83˜6.88 (m, 1H), 6.77 (s, 1H), 6.59 (s, 1H), 4.54 (t, J=4.8 Hz, 2H), 4.37 (t, J=4.8 Hz, 2H). MS (M+H)⁺: 289.

### Step 5—Synthesis of 5-(12-fluoro-4-oxo-6,7-dihydro-4H-pyrido[2′,1′:3,4]pyrazino[1,2-a]indol-2-yl)-2-(4-fluorophenyl)-N-methyl-6-(N-methylmethylsulfonamido)benzofuran-3-carboxamide

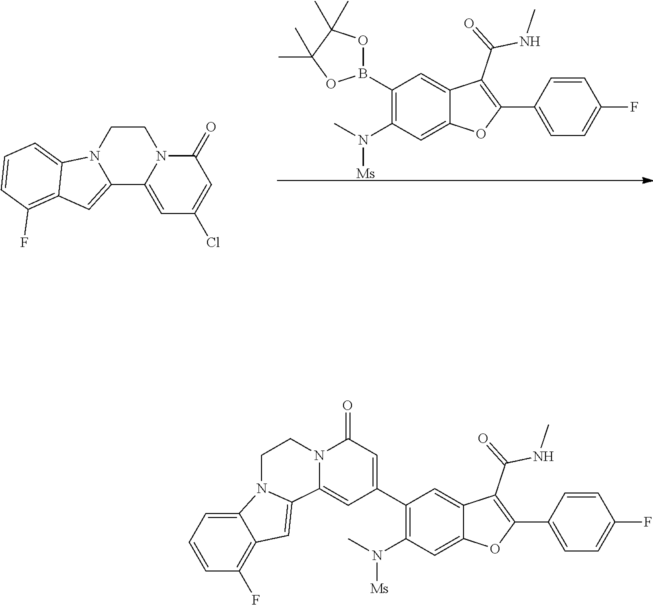 [SMILES: O=c1cc(Cl)cc2n1CCn1c-2cc2c(F)cccc21 ; CNC(=O)c1c(-c2ccc(F)cc2)oc2cc(N(C)S(C)(=O)=O)c(B3OC(C)(C)C(C)(C)O3)cc12 ; CNC(=O)c1c(-c2ccc(F)cc2)oc2cc(N(C)S(C)(=O)=O)c(-c3cc4n(c(=O)c3)CCn3c-4cc4c(F)cccc43)cc12]

To a mixture of 2-chloro-12-fluoro-6,7-dihydro-4H-pyrido[2′,1′:3,4]pyrazino[1,2-a]indol-4-one (50 mg, 0.17 mmol), 2-(4-fluorophenyl)-N-methyl-6-(N-methylmethylsulfonamido)-5-(4,4,5,5-tetramethyl-1,3,2-dioxaborolan-2-yl)benzofuran-3-carboxamide (80 mg, 0.17 mmol) and K₃PO₄.3H₂O (120 mg, 0.51 mmol) in 1,4-dioxane (4 mL) and water (0.5 mL) was added X-Phos (8 mg, 0.034 mmol) and Pd₂(dba)₃ (8 mg, 0.017 mmol) under nitrogen. The mixture was stirred at 90° C. for 6 hours and filtered through the celite pad. The filtrate was extracted with EtOAc, and the combined organic phase was washed with brine, dried over Na₂SO₄ and concentrated in vacuum. Finally the residue was purified by prep-TLC (PE:EA=1:2) to give 5-(12-fluoro-4-oxo-6,7-dihydro-4H-pyrido[2′,1′:3,4]pyrazino[1,2-a]indol-2-yl)-2-(4-fluorophenyl)-N-methyl-6-(N-methylmethylsulfonamido)benzofuran-3-carboxamide (35 mg, yield: 33%). ¹H-NMR (CDCl₃, 400 MHz) δ 7.93˜7.96 (m, 2H), 7.89 (s, 1H), 7.18˜7.26 (m, 3H), 7.12˜7.16 (m, 2H), 7.08 (s, 1H), 6.80˜6.85 (m, 1H), 6.60 (s, 1H), 5.98 (d, J=4.4 Hz, 1H), 4.61 (t, J=4.4 Hz, 2H), 4.41 (t, J=4.4 Hz, 2H), 3.22 (s, 3H), 3.00 (d, J=4.80 Hz, 3H), 2.78 (s, 3H). MS (M+H)⁺: 629.

### Example 31

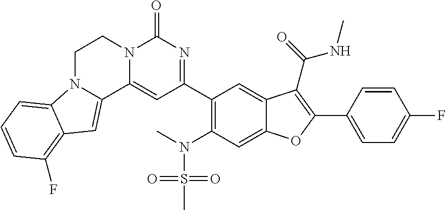 [SMILES: CNC(=O)c1c(-c2ccc(F)cc2)oc2cc(N(C)S(C)(=O)=O)c(-c3cc4n(c(=O)n3)CCn3c-4cc4c(F)cccc43)cc12]

### Step 1—Synthesis of tert-butyl 2-(2-chloro-6-(2-(4-fluorophenyl)-3-(methylcarbamoyl)-6-(N-methylmethylsulfonamido)benzofuran-5-pyrimidin-4-yl)-4-fluoro-1H-indole-1-carboxylate

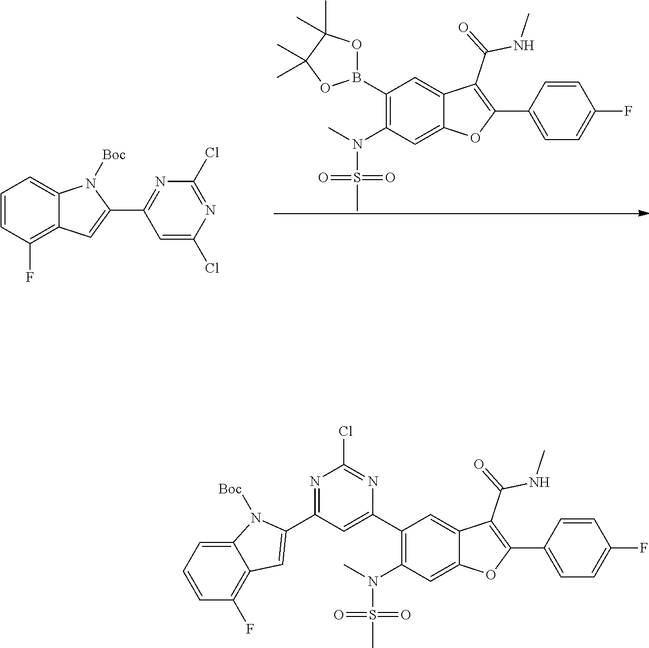 [SMILES: CC(C)(C)OC(=O)n1c(-c2cc(Cl)nc(Cl)n2)cc2c(F)cccc21 ; CNC(=O)c1c(-c2ccc(F)cc2)oc2cc(N(C)S(C)(=O)=O)c(B3OC(C)(C)C(C)(C)O3)cc12 ; CNC(=O)c1c(-c2ccc(F)cc2)oc2cc(N(C)S(C)(=O)=O)c(-c3cc(-c4cc5c(F)cccc5n4C(=O)OC(C)(C)C)nc(Cl)n3)cc12]

To a degassed mixture of tert-butyl 2-(2,6-dichloropyrimidin-4-yl)-4-fluoro-1H-indole-1-carboxylate (400 mg, 1.05 mmol, prepared from 2,4,6-trichloropyrimidine and (1-(tert-butoxycarbonyl)-4-fluoro-1H-indol-2-yl)boronic acid using similar procedure described), 2-(4-fluorophenyl)-N-methyl-6-(N-methylmethylsulfonamido)-5-(4,4,5,5-tetramethyl-1,3,2-dioxaborolan-2-yl)benzofuran-3-carboxamide (500 mg, 1.00 mmol) and K₃PO₄ (600 mg, 2.25 mmol) in 1,4-dioxane (10 mL) was added Pd(dppf)Cl₂ (50 mg) under N₂. The mixture was stirred at 100° C. for 2 hours. After the reaction mixture was cooled to RT, filtered and washed with EtOAc, the filtrate was washed with H₂O, brine, and dried over Na₂SO₄. After being concentrated, the residue was purified by column chromatography (DCM:MeOH=100:1) to give tert-butyl 2-(2-chloro-6-(2-(4-fluorophenyl)-3-(methylcarbamoyl)-6-(N-methylmethylsulfonamido)benzofuran-5-yl)pyrimidin-4-yl)-4-fluoro-1H-indole-1-carboxylate (690 mg, yield: 96.1%). ¹H-NMR (CDCl₃, 400 MHz) δ 8.22 (s, 1H), 7.93˜7.97 (m, 4H), 7.66 (s, 1H), 7.31˜7.37 (m, 1H), 7.20˜7.25 (m, 3H), 6.93˜6.98 (m, 1H), 5.94 (br s, 1H), 3.32 (s, 3H), 3.03 (d, J=4.8 Hz, 3H), 3.00 (s, 3H), 1.52 (s, 9H). MS (M+H)⁺: 722.

### Step 2—Synthesis of 5-(6-(4-fluoro-1H-indol-2-yl)-2-methoxypyrimidin-4-yl)-2-(4-fluorophenyl)-N-methyl-6-(N-methylmethylsulfonamido)benzofuran-3-carboxamide

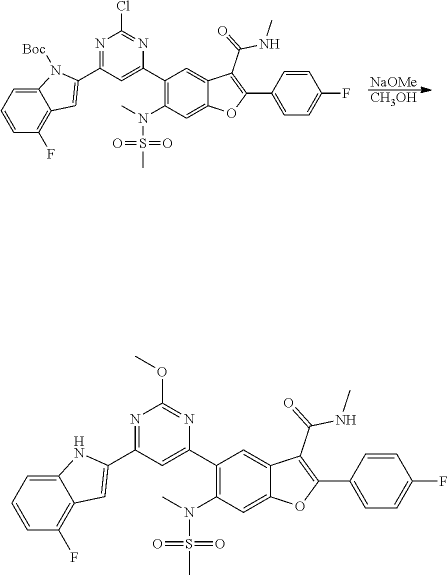

To a solution of tert-butyl 2-(2-chloro-6-(2-(4-fluorophenyl)-3-(methylcarbamoyl)-6-(N-methylmethylsulfonamido)benzofuran-5-yl)pyrimidin-4-yl)-4-fluoro-1H-indole-1-carboxylate (170 mg, 0.24 mmol) in MeOH (5 mL) was added NaOMe (40 mg, 0.74 mmol) and the mixture was stirred at 60° C. for 3 hours. Then the reaction mixture was adjusted to pH=7 with 1N HCl aq and extracted with EtOAc. After the combined organic phases were washed with brine, dried over Na₂SO₄, filtered and concentrated in vacuum, the residue was purified by prep-TLC (DCM:MeOH=40:1) to give 5-(6-(4-fluoro-1H-indol-2-yl)-2-methoxypyrimidin-4-yl)-2-(4-fluorophenyl)-N-methyl-6-(N-methylmethylsulfonamido)benzofuran-3-carboxamide (120 mg, yield: 82.5%). ¹H-NMR (CDCl₃, 400 MHz) δ 9.60 (br s, 1H), 8.15 (s, 1H), 7.92˜7.97 (m, 2H), 7.76 (s, 1H), 7.62 (s, 1H), 7.31 (s, 1H), 7.17˜7.22 (m, 4H), 6.76˜6.82 (m, 1H), 6.04 (br s, 1H), 4.14 (s, 3H), 3.29 (s, 3H), 3.02 (d, J=4.8 Hz, 3H), 2.97 (s, 3H). MS (M+H)⁺: 618.

### Step 3—Synthesis of 5-(6-(4-fluoro-1H-indol-2-yl)-2-methoxypyrimidin-4-yl)-2-(4-fluorophenyl)-N-methyl-6-(N-methylmethylsulfonamido)benzofuran-3-carboxamide

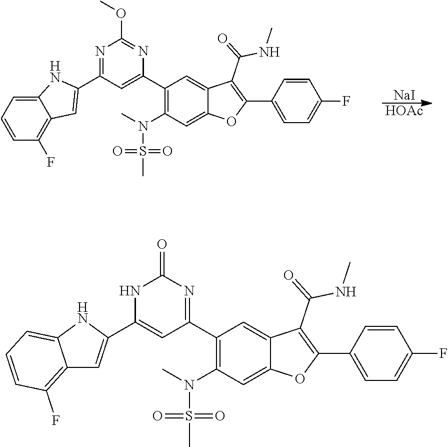 [SMILES: CNC(=O)c1c(-c2ccc(F)cc2)oc2cc(N(C)S(C)(=O)=O)c(-c3cc(-c4cc5c(F)cccc5[nH]4)nc(OC)n3)cc12 ; "NaI" ; CNC(=O)c1c(-c2ccc(F)cc2)oc2cc(N(C)S(C)(=O)=O)c(-c3cc(-c4cc5c(F)cccc5[nH]4)[nH]c(=O)n3)cc12 ; "HOAc"]

To a solution of 5-(6-(4-fluoro-1H-indol-2-yl)-2-methoxypyrimidin-4-yl)-2-(4-fluorophenyl)-N-methyl-6-(N-methylmethylsulfonamido)benzofuran-3-carboxamide (120 mg, 0.19 mmol) in HOAc (5 mL) was added NaI (150 mg, 1.00 mmol) and the mixture was stirred at 80° C. for 2 hours. After the mixture was concentrated in vacuum, the residue was suspended in water and extracted with EtOAc. The combined organic phases were washed with brine, dried over Na₂SO₄ and concentrated in vacuum. Finally the residue was purified by prep-HPLC to provide 5-(6-(4-fluoro-1H-indol-2-yl)-2-oxo-1,2-dihydropyrimidin-4-yl)-2-(4-fluorophenyl)-N-methyl-6-(N-methylmethylsulfonamido)benzofuran-3-carboxamide (100 mg, yield: 85.4%). ¹H-NMR (DMSO-d₆, 400 MHz) δ 12.26 (br s, 1H), 11.84 (br s, 1H), 8.46 (br s, 1H), 8.19 (s, 1H), 8.00˜8.04 (m, 2H), 7.92 (s, 1H), 7.41˜7.47 (m, 3H), 7.35 (d, J=8.0 Hz, 1H), 7.17˜7.23 (m, 2H), 6.8˜16.86 (m, 1H), 3.40 (s, 3H), 3.06 (s, 3H), 2.86 (d, J=4.4 Hz, 3H). MS (M+H)⁺: 604.

### Step 4—Synthesis of 5-(12-fluoro-4-oxo-6,7-dihydro-4H-pyrimido[6′,1′:3,4]pyrazino[1,2-a]indol-2-yl)-2-(4-fluorophenyl)-N-methyl-6-(N-methylmethylsulfonamido)benzofuran-3-carboxamide

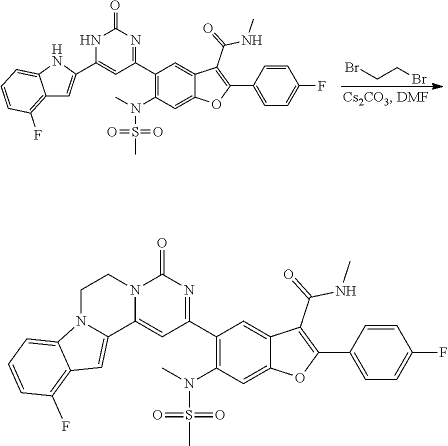

To a mixture of 5-(6-(4-fluoro-1H-indol-2-yl)-2-oxo-1,2-dihydropyrimidin-4-yl)-2-(4-fluorophenyl)-N-methyl-6-(N-methylmethylsulfonamido)benzofuran-3-carboxamide (50 mg, 0.08 mmol) and Cs₂CO₃ (60 mg, 0.18 mmol) in DMF (2 mL) was added dibromoethane (20 mg, 0.11 mmol) and the mixture was stirred at 80° C. for 2 hours. After the reaction mixture was concentrated in vacuo, the residue was suspended in water and extracted with EtOAc. The combined organic phases were washed with brine, dried over Na₂SO₄ and concentrated in vacuum. Finally the residue was purified by prep-HPLC to provide 5-(12-fluoro-4-oxo-6,7-dihydro-4H-pyrimido[6′,1′:3,4]pyrazino[1,2-a]indol-2-yl)-2-(4-fluorophenyl)-N-methyl-6-(N-methylmethylsulfonamido)benzofuran-3-carboxamide (15 mg, yield: 28.8%). ¹H-NMR (CDCl₃, 400 MHz) δ 8.21 (s, 1H), 8.00˜8.04 (m, 2H), 7.60 (s, 1H), 7.39 (s, 1H), 7.37 (s, 1H), 7.28˜7.34 (m, 1H), 7.17˜7.22 (m, 3H), 6.84˜6.89 (m, 1H), 6.16 (br s, 1H), 4.61˜4.63 (m, 2H), 4.46˜4.50 (m, 2H), 3.29 (s, 3H), 3.13 (s, 3H), 3.03 (d, J=4.8 Hz, 3H). MS (M+H)⁺: 630.

### Example 32

### Measuring Compound Inhibitory Potency

Measurement of inhibition by compounds was performed using the HCV replicon system. Several different replicons encoding different HCV genotypes or mutations were used. In addition, potency measurements were made using different formats of the replicon assay, including different ways of measurements and different plating formats. See Jan M. Vrolijk et al., A replicons-based bioassay for the measurement of interferons in patients with chronic hepatitis C, 110 J. Virological Methods 201 (2003); Steven S. Carroll et al., Inhibition of Hepatitis C Virus RNA Replication by 2′-Modified Nucleoside Analogs, 278(14) J. Biological Chemistry 11979 (2003). However, the underlying principles are common to all of these determinations, and are outlined below.

Stable neomycin phosphotransferase encoding replicons-harboring cell lines were used, so all cell lines were maintained under G418 selection prior to the assay. Potency was determined using a cell ELISA assay with an antibody to the replicons encoded NS3/4a protease. See Caterina Trozzi et al., In Vitro Selection and Characterization of Hepatitis C Virus Serine Protease Variants Resistant to an Active-Site Peptide Inhibitor, 77(6) J. Virol. 3669 (2003). To initiate an assay, replicon cells were plated in the presence of a dilution series of test compound in the absence of G418. Typically, the assays were performed in a 96-well plate formate for manual operation, or a 384-well plate format for automated assay. Replicon cells and compound were incubated for 96 hours. At the end of the assay, cells were washed free of media and compound, and the cells were then lysed. RNA was quantified indirectly through detection of replicon-encoded NS3/4A protein levels, through an ELISA-based assay with an antibody specific for NS3/4A. IC₅₀ determinations were calculated as a percentage of a DMSO control by fitting the data to a four-parameter fit function and the data obtained is provided in the table below.

Data for selected compounds of the present invention was obtained for genotypes 1a and 1b using this method and is provided in the table below:

| Compound | 1a | 1b |
|---|---|---|
| No. | IC₅₀ (nM) | IC₅₀ (nM) |
| 1 | 3.616 | 3.885 |
| 2 | 2.474 | 2.433 |
| 3 | 2.977 | 5.365 |
| 4 | 5.712 | 6.812 |
| 5 | 5.816 | 3.714 |
| 6 | 16.47 | 8.378 |
| 7 | 1.553 | 1.1 |
| 8 | 6.452 | 5.948 |
| 9 | 13.45 | 4.16 |
| 10 | 22.69 | 10.13 |
| 11 | 3.843 | 2.674 |
| 12 | 3.283 | 1.671 |
| 13 | 4.516 | 6.591 |
| 14 | 3.552 | 4.433 |
| 15 | 238.1 | 81.14 |
| 16 | 1.379 | 1.381 |
| 17 | 2.289 | 4.265 |
| 18 | 3.146 | 5.016 |
| 19 | 2.483 | 3.072 |
| 20 | 2.905 | 5.913 |
| 21 | 13.69 | 5.989 |
| 22 | 354.1 | 192.9 |
| 23 | 492.1 | 212.9 |
| 24 | 80.82 | 35.5 |
| 25 | 72.51 | 38.3 |
| 26 | 129.1 | 29.71 |
| 27 | 6.865 | 4.466 |
| 28 | 115.8 | 17.98 |
| 29 | 109.7 | 31.76 |
| 30 | 3.521 | 19.03 |
| 31 | 3.481 | 6.947 |

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A compound having the formula: 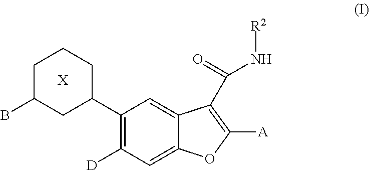
or a pharmaceutically acceptable salt thereof,
wherein:
X is 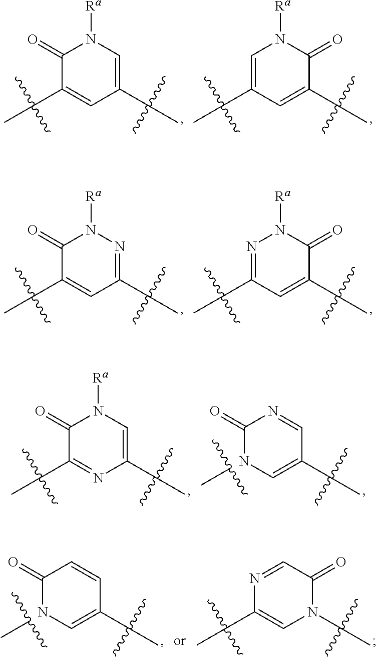
Bis a) Ar; or
b) —C(═O)NHCR⁵R⁶Ar; or
X together with B is 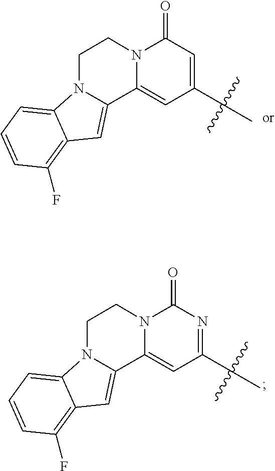 [SMILES: *c1cc2n(c(=O)c1)CCn1c-2cc2c(F)cccc21 ; *c1cc2n(c(=O)n1)CCn1c-2cc2c(F)cccc21]
Ar is an aromatic ring system selected from:
5-6 membered monocyclic ring with 0, 1, or 2 N ring atoms, optionally substituted with halo or fluorophenyl; and
(ii) 9-10 membered bicyclic rings with 0, 1, 2 or 3 heteroatom ring atoms selected from N and O, which is optionally substituted with 1 or 2 substituents independently selected from C₁-C₆ alkyl, F, cyano, and alkylalkoxy;
A is fluorophenyl;
D is absent or NR³SO₂R⁴;
R^{a} is C₁-C₆ alkyl or C₁-C₆ haloalkyl;
R² is C₁-C₆ alkyl;
R³ is C₁-C₆ alkyl;
R⁴ is C₁-C₆ alkyl;
R⁵ is hydrogen, C₁-C₆ alkyl, or C₁-C₆ hydroxyalkyl;
R⁶ is hydrogen; or
R⁵ and R⁶ together with the carbon to which they are attached form cyclopropyl.

2. The compound of claim 1, or a pharmaceutically acceptable salt thereof, wherein R², R³ and R⁴ are methyl.

3. The compound of claim 2, or a pharmaceutically acceptable salt thereof, wherein D is N(CH₃)SO₂CH₃.

4. The compound of claim 2, or a pharmaceutically acceptable salt thereof, wherein each halo is F.

5. The compound of claim 4, or a pharmaceutically acceptable salt thereof, wherein R^{a} is methyl or —CHF₂.

6. The compound of claim 5, or a pharmaceutically acceptable salt thereof, wherein R⁵ is hydrogen, methyl or —CH₂OH, or
R⁵ and R⁶ together with the carbon to which they are attached form cyclopropyl.

7. The compound of claim 1, or a pharmaceutically acceptable salt thereof, having the formula:

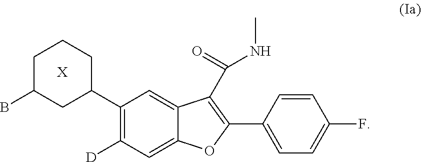

8. The compound of claim 7, or a pharmaceutically acceptable salt thereof, wherein B is
fluorophenyl;
pyrazole substituted with fluorophenyl;
—C(═O)NHCH(CH₃)-fluorophenyl;
—C(═O)NHCH(CH₂OH)-fluorophenyl;
—C(═O)NHCH₂-fluoropyridine;
—C(═O)NH-cyclopropyl-phenyl; or
—C(═O)NH-cyclopropyl-fluorophenyl.

9. The compound of claim 7, or a pharmaceutically acceptable salt thereof, wherein B is
indole substituted 1 or 2 substituents selected from H, F, cyano, —CH₂CH₂OCH₃;
benzooxazole;
isoindolinone substituted with F;
furopyridine;
oxaxolopyridine;
pyrrolopryidine;
naphthalene; or
—C(═O)NH-cyclopropyl-naphthyridine.

10. The compound of claim 1 which is any one of 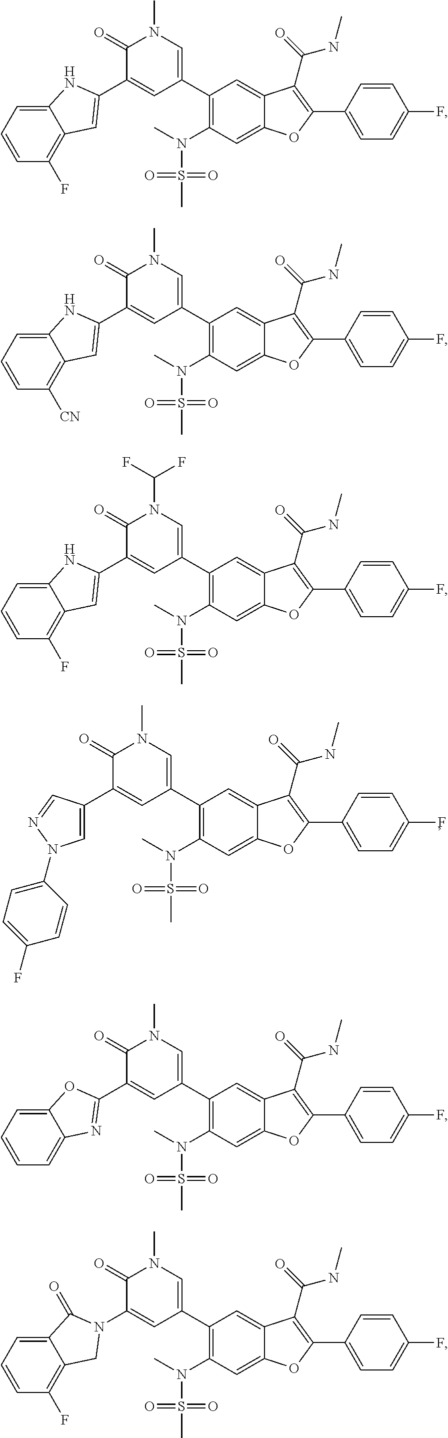 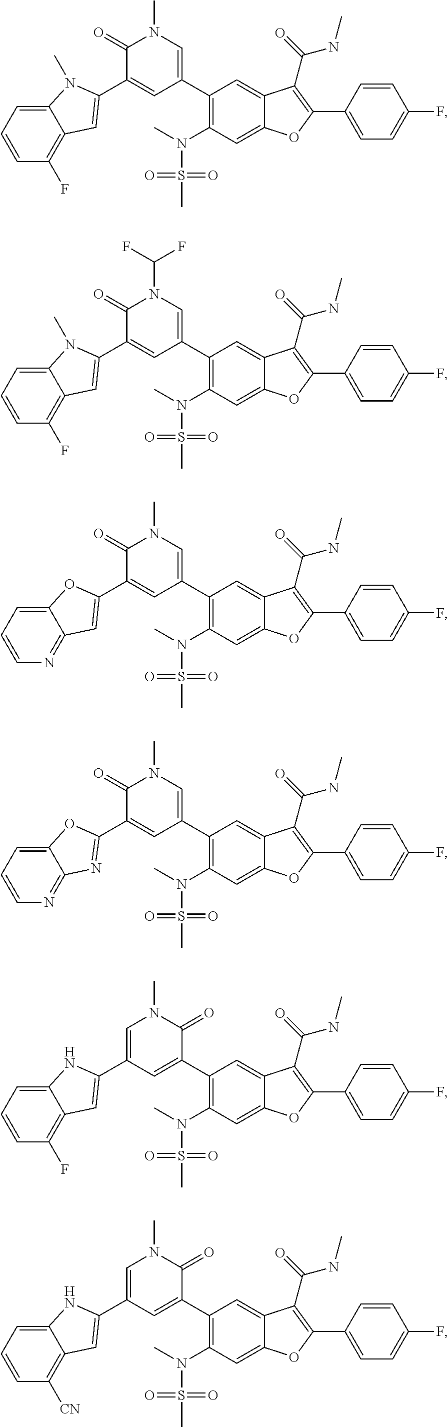 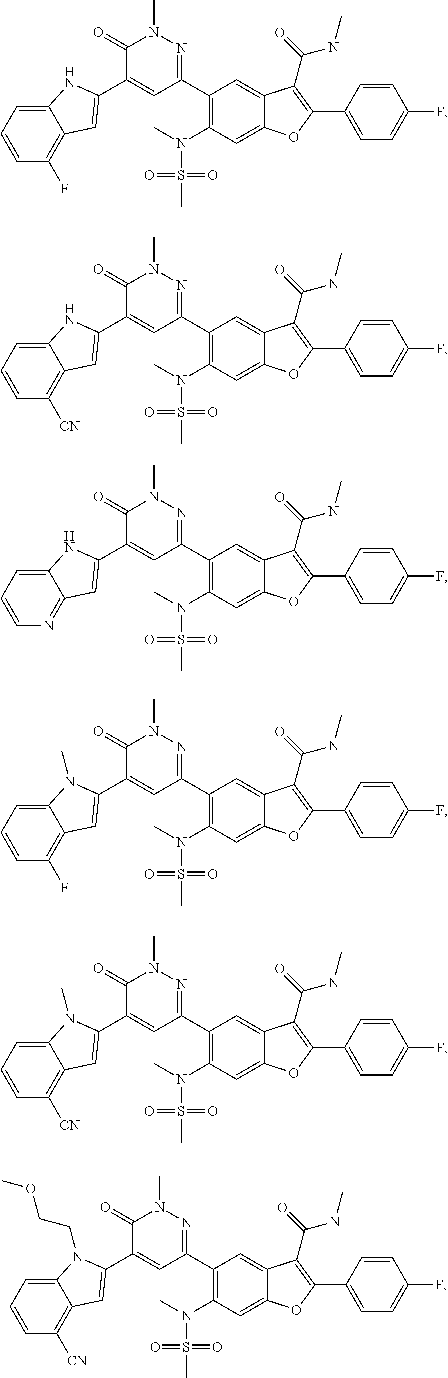 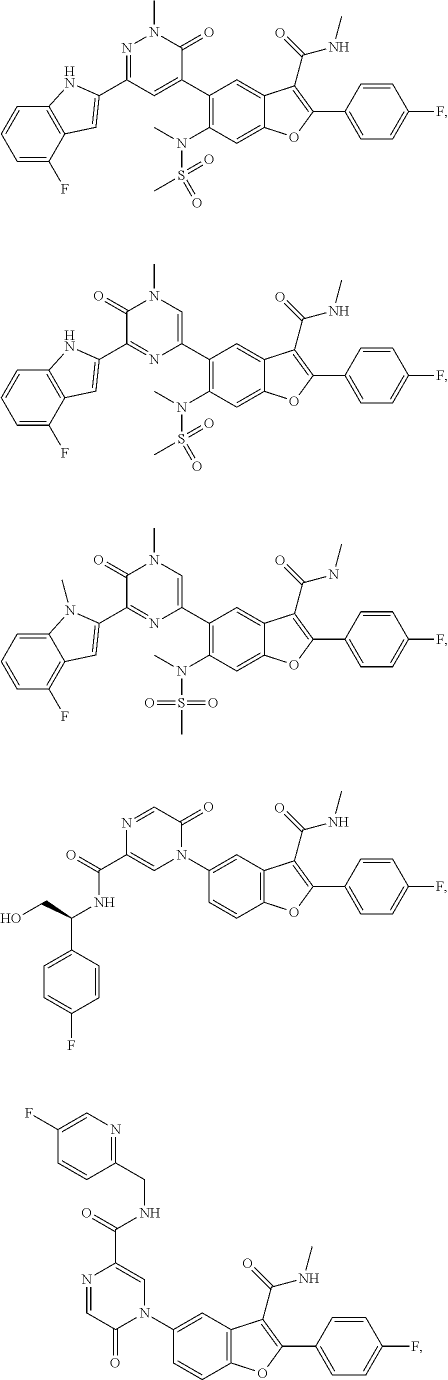 [SMILES: CNC(=O)c1c(-c2ccc(F)cc2)oc2cc(N(C)S(C)(=O)=O)c(-c3cc(-c4cc5c(F)cccc5[nH]4)nn(C)c3=O)cc12 ; CNC(=O)c1c(-c2ccc(F)cc2)oc2cc(N(C)S(C)(=O)=O)c(-c3cn(C)c(=O)c(-c4cc5c(F)cccc5[nH]4)n3)cc12 ; CNC(=O)c1c(-c2ccc(F)cc2)oc2cc(N(C)S(C)(=O)=O)c(-c3cn(C)c(=O)c(-c4cc5c(F)cccc5n4C)n3)cc12 ; CNC(=O)c1c(-c2ccc(F)cc2)oc2ccc(-n3cc(C(=O)N[C@H](CO)c4ccc(F)cc4)ncc3=O)cc12 ; CNC(=O)c1c(-c2ccc(F)cc2)oc2ccc(-n3cc(C(=O)NCc4ccc(F)cn4)ncc3=O)cc12] 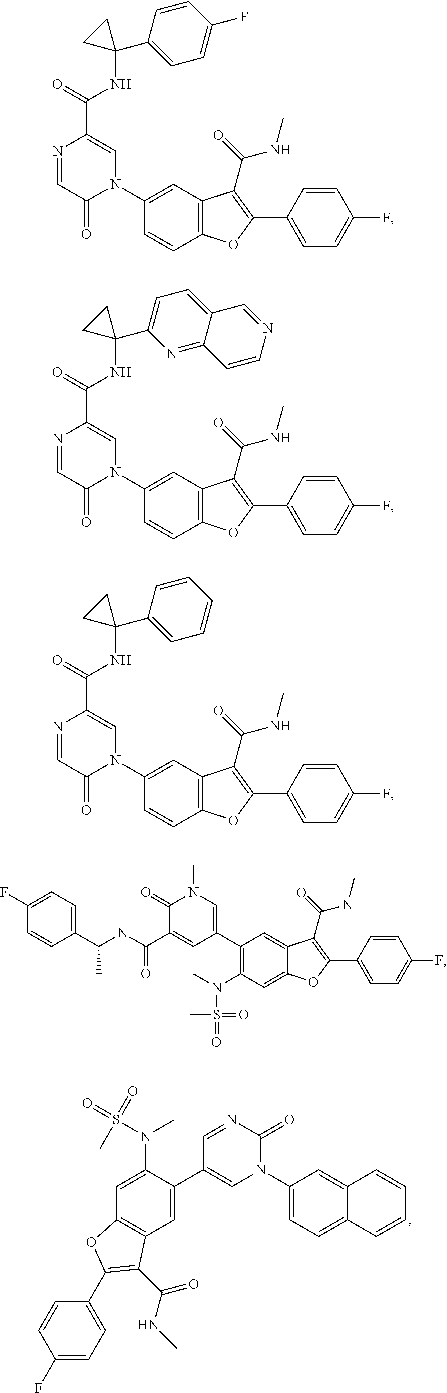 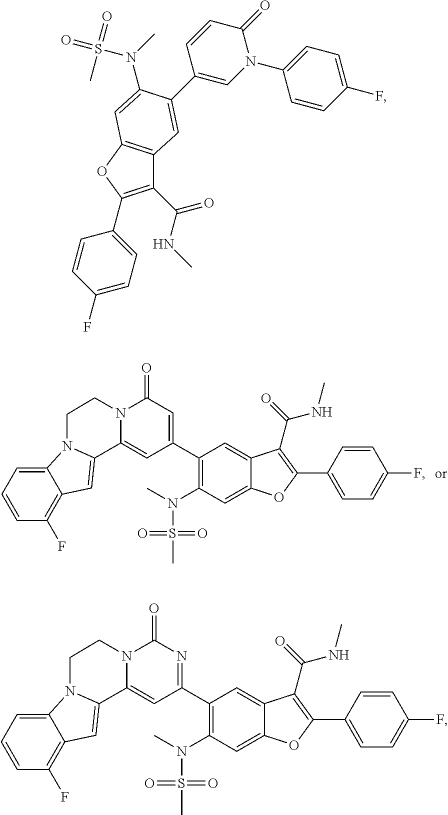 [SMILES: CNC(=O)c1c(-c2ccc(F)cc2)oc2cc(N(C)S(C)(=O)=O)c(-c3ccc(=O)n(-c4ccc(F)cc4)c3)cc12 ; CNC(=O)c1c(-c2ccc(F)cc2)oc2cc(N(C)S(C)(=O)=O)c(-c3cc4n(c(=O)c3)CCn3c-4cc4c(F)cccc43)cc12 ; CNC(=O)c1c(-c2ccc(F)cc2)oc2cc(N(C)S(C)(=O)=O)c(-c3cc4n(c(=O)n3)CCn3c-4cc4c(F)cccc43)cc12]
or a pharmaceutically acceptable salt thereof.

11. A pharmaceutical composition comprising (i) a pharmaceutically acceptable carrier and (ii) an effective amount of the compound of claim 1 or a pharmaceutically acceptable salt thereof.

12. The pharmaceutical composition of claim 11, further comprising a second therapeutic agent selected from the group consisting of HCV antiviral agents, immunomodulators, and anti-infective agents.

13. The pharmaceutical composition of claim 12, wherein the second therapeutic agent is selected from the group consisting of HCV NS3 and NS3/4A protease inhibitors, HCV NS5A inhibitors and HCV NS5B polymerase inhibitors.

14-15. (canceled)

16. A method of treating a patient infected with HCV, the method comprising administering to the patient the compound of claim 1, or a pharmaceutically acceptable salt thereof, in an amount effective to treat infection by HCV in the patient.

17. The method of claim 16, further comprising administering to said patient an effective amount of at least one second therapeutic agent selected from the group consisting of HCV NS3 and NS3/4A protease inhibitors, HCV NS5A inhibitors and HCV NS5B polymerase inhibitors.
